(19) United States
(12) Patent Application Publication
(10) Pub. No.: US 2024/0423549 A1
(43) Pub. Date: Dec. 26, 2024

(54) SYSTEMS AND METHODS FOR AUTOMATICALLY CLASSIFYING WIDE COMPLEX TACHYCARDIAS (WCTS)

(71) Applicant: Washington University, St. Louis, MO (US)

(72) Inventor: Adam May, St. Louis, MO (US)

(21) Appl. No.: 18/706,109

(22) PCT Filed: Nov. 1, 2022
(86) PCT No.: PCT/US2022/048618

§ 371 Date: Apr. 30, 2024

Related U.S. Application Data
(60) Provisional application No. 63/274,096, filed on Nov. 1, 2021.

(51) Int. Cl. A61B 5/00; A61B 5/355; A61B 5/363; A61B 5/364; A61B 5/366; G16H 20/40

(52) U.S. Cl. CPC A61B 5/7267; A61B 5/355; A61B 5/363; A61B 5/364; A61B 5/366; A61B 5/7253; A61B 5/7275; G16H 20/40

(57) ABSTRACT

Systems and computer-aided methods for automatically classifying a wide complex tachycardia (WCT) pattern of a subject are disclosed that include receiving ECG data indicative of the WCT pattern, transforming the ECG data into at least one engineered feature using at least one transform, and transforming the at least one engineered feature into a classification of the WCT pattern using a predictive model. The classification of the WCT pattern may be transformed into a treatment recommendation.

### CROSS-REFERENCE TO RELATED APPLICATIONS

This application claims priority from U.S. Provisional Application Ser. No. 63/274,096 filed on Nov. 1, 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to computer-aided systems and methods of automatically classifying wide complex tachycardias (WCTs).

### BACKGROUND OF THE INVENTION

Accurate and timely wide QRS complex tachycardia (WCT) differentiation into ventricular tachycardia (VT) and supraventricular wide QRS tachycardia (SWCT) by way of 12-lead electrocardiogram (ECG) interpretation is a critical responsibility for front-line medical providers. However, despite its undeniable clinical importance, the successful distinction of VT from SWCT continues to be a difficult task in clinical practice, especially for clinicians lacking sufficient ECG interpretation expertise or practiced proficiency using traditional ECG interpretation methods. Inopportune occasions that call attention to this limitation are commonplace, especially among clinicians who regularly encounter patients with VT or SWCT within demanding and high-stress clinical environments (e.g., emergency departments and intensive care units).

Although numerous WCT differentiation criteria and algorithms are readily available for clinical use, each universally possesses inherent and unavoidable limitations relating to their manual application. In genuine “real life” clinical circumstances, front-line clinicians must do the following to accurately differentiate WCTs by way of 12-lead ECG interpretation: (i) acquire and then visually inspect a properly recorded and well-timed 12-lead ECG that sufficiently ‘captures’ the WCT event, (ii) summon forth or precisely recall one or more of the available WCT differentiation criteria or algorithms, and (iii) sedulously implement the chosen WCT differentiation method(s) without error, even while under duress.

Given the complexity of the task, accurate and timely WCT differentiation is often thwarted by the practical limitations of manual methods, and clinical mistakes may occur despite clinicians' well-intentioned attempts to differentiate WCTs accurately. As such, front-line clinicians who arrive at inaccurate or delayed diagnoses may unintentionally escort WCT patients toward missed therapeutic opportunities, harmful medical therapies, or unsafe clinical decisions.

Given the inherent challenges associated with manually-operated WCT differentiation methods, automated solutions to distinguish VT and SWCT have been proposed, including the WCT Formula [2019], the VT Prediction Model [2020], and the WCT Formula II [2020]. The fundamental characteristic of each proposed method is the integration of mathematically-formulated parameters (e.g., frontal percent amplitude change [PAC] [%] or horizontal percent time-voltage area change [PTVAC] [%]) each directly derived from computerized ECG measurement data, into an automatic binary classification model. These automated WCT differentiation models can provide an effective means to differentiate WCTs accurately once they are integrated into existing computerized ECG interpretation software systems. However, given that each automated WCT differentiation method requires the simultaneous analysis of paired WCT and baseline ECG data, existing automated methods cannot be applied to patients lacking previously recorded and archived baseline ECGs.

### SUMMARY OF THE INVENTION

Among the various aspects of the present disclosure is the provision of systems and methods for classifying a wide complex tachycardia (WCT) pattern of a subject.

In one aspect, a computer-aided method of classifying a wide complex tachycardia (WCT) pattern of a subject is disclosed that includes receiving ECG data indicative of the WCT pattern, transforming the ECG data into at least one engineered feature using at least one transform, and transforming the at least one engineered feature into a classification of the WCT pattern using a model. In some aspects, transforming the at least one engineered feature into the classification of the WCT pattern further includes assigning the classification comprising one of a ventricular tachycardia (VT), a supraventricular wide complex tachycardia (SWCT), a probability of a VT, and a probability of an SWCT. In some aspects, receiving the ECG data indicative of the WCT pattern includes receiving ECG data from an ECG device that may be a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automatic implantable cardioverter defibrillator (AICD), an automated external defibrillator (AED), and any combination thereof. In some aspects, receiving the ECG data indicative of the WCT pattern includes receiving ECG data from the 12-lead ECG device. In some aspects, transforming the ECG data into at least one engineered feature further includes transforming, using the computing device, the ECG data into at least one of percent monophasic time-voltage area (PMonoTVA), percent monophasic amplitude (PMonoAmp), frontal percent amplitude change (Frontal PAC), horizontal percent amplitude change (Horizontal PAC), and WCT QRS duration. In some aspects, transforming the ECG data into the at least one engineered feature using at least one transform further includes transforming the ECG data using automated data analysis software and receiving, using the computing device, at least one engineered feature from the automated data analysis software. In some aspects, transforming the ECG data into at least one engineered feature using at least one transform further includes transforming the ECG data into PMonoTVA using a first transform

$$PMonoTVA = \frac{( Monophasic TVA )}{( Monophasic TVA ) + ( Multiphasic TVA )} \times 100$$

wherein Monophasic TVA comprises a summation of all QRS time-voltage areas from all monophasic QRS complexes from the ECG data and Multiphasic TVA comprises a summation of all QRS time-voltage areas from all multiphasic QRS complexes from the ECG data. In some aspects, wherein transforming the ECG data into at least one engineered feature using at least one transform further includes transforming the ECG data into PMonoAmp using a second transform

$$PMonoAmp = \frac{( Monophasic amplitude )}{( Monophasic amplitude ) + ( Multiphasic amplitude )} \times 1$$

wherein Monophasic amplitude comprises a summation of all QRS amplitudes from all monophasic QRS complexes from the ECG data and Multiphasic amplitude comprises a summation of all QRS amplitudes from all multiphasic QRS complexes from the ECG data. In some aspects, transforming at least one engineered feature into a classification of the WCT pattern using a model further includes using a machine learning model comprising one of a logistic regression model, an artificial neural network, a random forest model, and a support vector machine. In some aspects, transforming the at least one engineered feature into a classification of the WCT pattern using a model further includes using the logistic regression model. In some aspects, using the logistic regression model further includes transforming the at least one engineered feature into a weighted sum of predictors X_{β} using the equation X_{β}=β₀+β₁X₁+β₂X₂ wherein β₀, β₁, and β₂ are constant weighting factors, X₁ is PMonoTVA, and X₂ is WCT QRS Duration; and calculating the probability of the VT (P_{VT}) using the equation

$${P}_{VT} = \frac{{e}^{{X}_{β}}}{1 + {e}^{{X}_{β}}} .$$

In some aspects, the method further includes receiving baseline ECG data indicative of a baseline cardiac pattern and transforming the baseline ECG data and the ECG data into the at least one engineered feature using at least one transform. In some aspects, transforming the baseline ECG data and the WCT ECG data into at least one engineered feature further includes transforming the baseline ECG data and the ECG data into at least one of QRS axis change, T axis change, Frontal PAC, and Horizontal PAC. In some aspects, using the logistic regression model further includes transforming the at least one engineered feature into a weighted sum of predictors X_{β} using the equation X_{β}=β₀+β₁X₁+β₂X₂+β₃X₃+β₄X₄+β₅X₅+β₆X₆ wherein β₀, β₁, β₂, β₃, β₄, β₅, and β₆ are constant weighting factors, X₁ is PMonoTVA, X₂ is WCT QRS Duration, X₃ is T Axis change, X₄ is Frontal PAC, and X₆ is Horizontal PAC; and calculating the probability of the VT (P_{VT}) using the equation

$${P}_{VT} = \frac{{e}^{{X}_{β}}}{1 + {e}^{{X}_{β}}} .$$

In some aspects, wherein transforming the at least one engineered feature into the classification of the WCT pattern further includes assigning the classification of VT if P_{VT} is at least equal to a predetermined threshold value, and assigning the classification of SWCT if P_{VT} is less than the predetermined threshold value. In some aspects, the predetermined threshold value ranges from 0% to 100%. In some aspects, the predetermined threshold value comprises one of 1%, 10%, 25%, 50%, 75%, 90%, 95%, and 99%. In some aspects, the method further includes transforming the assigned classification of the WCT pattern into a treatment recommendation using at least one treatment rule. In some aspects, the at least one treatment rule is selected from one of recommending a shock delivery to the heart of the subject if the assigned classification is VT and recommending no shock delivery if the assigned classification is SWCT.

In another aspect, a system for classifying a wide complex tachycardia (WCT) pattern of a subject is disclosed that includes a computing device with at least one processor configured to receive ECG data indicative of the WCT pattern, transform the ECG data into at least one engineered feature using at least one transform, and transform the at least one engineered feature into a classification of the WCT pattern using a model. In some aspects, the classification comprises one of a ventricular tachycardia (VT), a supraventricular wide complex tachycardia (SWCT), a probability of a VT, and a probability of an SWCT. In some aspects, the ECG device includes one of a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automated external defibrillator (AED), an automatic implantable cardioverter defibrillator (AICD), and any combination thereof. In some aspects, the ECG device is the 12-lead ECG device. In some aspects, the at least one engineered feature includes at least one of percent monophasic time-voltage area (PMonoTVA), percent monophasic amplitude (PMonoAmp), frontal percent amplitude change (Frontal PAC), horizontal percent amplitude change (Horizontal PAC), and WCT QRS duration. In some aspects, the at least one processor is further configured to receive a portion of the engineered features from automated data analysis software configured to transform the ECG data into the portion of the engineered features. In some aspects, the at least one processor is further configured to transform the ECG data into PMonoTVA using a first transform

$$PMonoTVA = \frac{( Monophasic TVA )}{( Monophasic TVA ) + ( Multiphasic TVA )} \times 100$$

wherein Monophasic TVA is a summation of all QRS time-voltage areas from all monophasic QRS complexes from the ECG data and Multiphasic TVA is a summation of all QRS time-voltage areas from all multiphasic QRS complexes from the ECG data. In some aspects, the at least one processor is further configured to transform the ECG data into PMonoAmp using a second transform

$$PMonoAmp = \frac{( Monophasic amplitude )}{( Monophasic amplitude ) + ( Multiphasic amplitude )} \times 100$$

wherein Monophasic amplitude is a summation of all QRS amplitudes from all monophasic QRS complexes from the ECG data and Multiphasic amplitude is a summation of all QRS amplitudes from all multiphasic QRS complexes from the ECG data. In some aspects, the at least one processor is further configured to transform the least one engineered feature into a classification of the WCT pattern using a machine learning model that includes one of a logistic regression model, an artificial neural network, a random forest model, and a support vector machine. In some aspects, the machine learning model is the logistic regression model. In some aspects, the at least one processor is further configured to transform the least one engineered feature into a classification of the WCT pattern using the logistic regression model by transforming the at least one engineered feature into a weighted sum of predictors X_{β} using the equation X_{β}=β₀+β₁X₁+β₂X₂ wherein β₀, β₁, and β₂ are constant weighting factors, X₁ is PMonoTVA, and X₂ is WCT QRS Duration; and calculating the probability of the VT (P_{VT}) using the equation

$${P}_{VT} = \frac{{e}^{{X}_{β}}}{1 + {e}^{{X}_{β}}} .$$

In some aspects, the at least one processor is further configured to receive baseline ECG data indicative of a baseline cardiac pattern and transform the baseline ECG data and the WCT ECG data into at least one engineered feature using at least one transform. In some aspects, the at least one engineered feature further includes at least one of QRS axis change, T axis change, Frontal PAC, and Horizontal PAC. In some aspects, the at least one processor is further configured to transform the least one engineered feature into a classification of the WCT pattern using the logistic regression model by transforming the at least one engineered feature into a weighted sum of predictors X_{β} using the equation X_{β}=β₀+β₁X₁+β₂X₂+β₃X₃+β₄X₄+β₅X₅+β₆X₆ wherein β₀, β₁, β₂, β₃, β₄, β₅, and β₆ are constant weighting factors, X₁ is PMonoTVA, X₂ is WCT QRS Duration, X₃ is T Axis change, X₄ is Frontal PAC, and X₆ is Horizontal PAC; and calculating the probability of the VT (P_{VT}) using the equation

$${P}_{VT} = \frac{{e}^{{X}_{β}}}{1 + {e}^{{X}_{β}}} .$$

In some aspects, the at least one processor is further configured to transform the least one engineered feature into a classification of the WCT pattern using the logistic regression model by assigning the classification of VT if P_{VT} is at least equal to a predetermined threshold value, and assigning the classification of SWCT if P_{VT} is less than the predetermined threshold value. In some aspects, the predetermined threshold value ranges from 40% to 60%. In some aspects, the predetermined threshold value comprises one of 1%, 10%, 25%, 50%, 75%, 90%, 95%, and 99%. In some aspects, the at least one processor is further configured to transform the assigned classification of the WCT pattern into a treatment recommendation using at least one treatment rule. In some aspects, the at least one treatment rule is selected from one of recommending a shock delivery to the heart of the subject if the assigned classification is VT, and recommending no shock delivery if the assigned classification is SWCT. In some aspects, the system further includes the ECG device operatively coupled to the computing device. In some aspects, the system further includes a treatment device operatively coupled to the computing device, the treatment device configured to perform the shock delivery to the heart of the subject in response to the treatment recommendation of shock delivery.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a flow diagram depicting the process of WCT ECG cohort selection used in Example 1 below; ECG denotes electrocardiogram and WCT denotes wide complex tachycardia.

FIG. 6 is a schematic representation of a stereotypical QRS complex and its measurable components provided by computerized ECG interpretation software. QRS amplitude (μV) represents the vertical height of positive (r/R and r′/R′) and negative (q/QS, s/S, and s′/S′) QRS waveforms. QRS time-voltage area (TVA) (μV·ms) represents the “area” enveloped by an individual QRS complex waveform above (r/R and r′/R′) or below (q/QS, s/S, and s′/S′) the isoelectric baseline. TVA denotes time-voltage area, PA denotes p wave amplitude, and TA denotes T wave amplitude.

FIG. 7 is a flow chart illustrating the calculation of PMonoTVA, an engineered feature derived from a measured QRS waveform TVA (time-voltage area) (μV·ms) of the dominant QRS complex template within each lead of the 12-lead ECG. ECG refers to electrocardiogram, PMonoTVA refers to percent monophasic time voltage area, TVA refers to time voltage area, and WCT refers to wide complex tachycardia.

FIG. 8 is a flow chart illustrating the calculation of PMonoAmp, an engineered feature derived from a measured QRS waveform amplitude (μV) of the dominant QRS complex template within each lead of the 12-lead ECG. ECG refers to electrocardiogram, PMonoAmp refers to percent monophasic amplitude, and WCT refers to wide complex tachycardia.

FIG. 9 is an illustration of the logistic regression structure of WCT Model I. WCT engineered features (Xₓ) are assigned beta coefficients (βₓ) according to their effects on the binary outcome, which is either a VT or SWCT classification of WCT ECG data. The “constant” term (β₀) represents the y-intercept for the least-squares regression line. The weighted sum predictor (X_{β}) or VT probability (P) is calculated after integrating WCT engineered feature (Xₓ) values derived from paired WCT and baseline ECG data. PMonoTVA refers to percent monophasic time voltage area, SWCT refers to supraventricular tachycardia, VT refers to ventricular tachycardia, and WCT refers to wide complex tachycardia.

FIG. 10 is an illustration of the logistic regression structure of WCT Model II. WCT engineered features (Xₓ) are assigned beta coefficients (βₓ) according to their effects on the binary outcome which is either a VT or SWCT classification of WCT ECG data. The “constant” term (β₀) represents the y-intercept for the least-squares regression line. The weighted sum predictor (X_{β}) or VT probability (P) is calculated after integrating WCT engineered feature (Xₓ) values derived from paired WCT and baseline ECG data. PMonoTVA refers to percent monophasic time voltage area, PAC refers to percent amplitude change, PTVAC refers to percent time-voltage area change, SWCT refers to supraventricular tachycardia, VT refers to ventricular tachycardia, and WCT refers to wide complex tachycardia.

FIG. 11 is a box-plot summarizing the median and proportional distribution of PMonoTVA (%). PMonoTVA refers to percent monophasic time voltage area, SWCT refers to supraventricular wide complex tachycardia, and VT refers to ventricular tachycardia.

FIG. 12 is a box-plot summarizing the median and proportional distribution of PMonoAmp (%). PMonoAmp, percent monophasic amplitude; SWCT, supraventricular wide complex tachycardia; VT, ventricular tachycardia.

FIG. 13 is a graph comparing the receiver operating characteristic (ROC) curves for the WCT Model I and WCT Model II.

FIG. 14 contains a series of schematic diagrams illustrating the development of monophasic QRS complexes due to VT. The left panels demonstrate the development of a negative monophasic QRS complex in ECG lead aVF. The right panels demonstrate the development of a positive monophasic QRS complex in lead V1. Each panel series (A to B to C) demonstrates the progression of ventricular depolarization wavefronts that spread uniformly from the site-of-origin (SOO). Embedded yellow arrows depict the directionality of ventricular depolarization. Red shading represents depolarized myocardium. LV refers to left ventricle, RV refers to right ventricle, and SOO refers to site-of-origin.

FIG. 15 is a schematic diagram illustrating exemplary monophasic QRS waveform configurations above (‘R’ complex) or below (‘QS’ complex) the isoelectric baseline. Color-shaded regions (blue shading: above baseline; orange shading: below baseline) demonstrate the time-voltage areas (TVAs) for selected examples of monophasic QRS complexes. TVA refers to time-voltage area.

FIG. 16 contains a series of schematic diagrams illustrating the development of exemplary multiphasic QRS complexes due to SWCT with aberrancy. The left panels demonstrate the creation of multiphasic QRS complexes in leads V1 and V6 due to RBBB. The right panels demonstrate the creation of a multiphasic QRS complex in leads V1 and V6 due to LBBB. Each panel series (A to B to C) demonstrates the progression of ventricular depolarization wavefronts that stereotypically occur for RBBB (left panels) and LBBB (right panels). Embedded yellow arrows depict the directionality and intensity of the mean electrical vector during ventricular depolarization. Red shading represents depolarized myocardium. Ao refers to aorta, LA refers to left atrium, LV refers to left ventricle, RA refers to right atrium, RV refers to right ventricle, LBBB refers to left bundle branch block, and RBBB refers to right bundle branch block.

FIG. 17 is a schematic diagram illustrating exemplary multiphasic QRS waveform configurations above or below the isoelectric baseline. Color-shaded regions (blue shading: above baseline; orange shading: below baseline) demonstrate the time-voltages areas (TVAs) for examples of a multiphasic QRS complex. TVA, time-voltage area.

FIG. 18 is a flowchart illustrating the coordinated application of WCT Model I and WCT Model II for WCT rhythm classification (VT or SWCT). ECG refers to electrocardiogram, SWCT refers to supraventricular wide complex tachycardia, VT refers to ventricular tachycardia, and WCT refers to wide complex tachycardia.

FIG. 19 is a flowchart illustrating the coordinated application of WCT Model I and WCT Model II for VT probability estimation. ECG refers to electrocardiogram, SWCT refers to supraventricular wide complex tachycardia, VT refers to ventricular tachycardia, and WCT refers to wide complex tachycardia.

FIG. 20 is a flowchart illustrating the frontal PAC (%) calculation. Frontal PAC (%) is composed of measured QRS waveform amplitudes (μV) derived from selected ECG leads within the frontal plane. LeadX denotes the selected individual ECG leads within the frontal (aVR, aVL, aVF) ECG plane. Positive Amplitude (PA) is the sum of measured QRS waveform amplitudes above the isoelectric baseline (r/R and r′/R′) in a single ECG lead. Negative Amplitude (NA) is the sum of measured QRS waveform amplitudes below the isoelectric baseline (q/QS, s/S, and s′/S′) in a single ECG lead. Total Baseline Amplitude (TBA) is the sum of PA and NA within the selected individual ECG leads of the baseline ECG. Baseline Amplitude (BA) is the summation of TBAs from the selected ECG leads in the frontal (aVR, aVL, aVF) ECG plane. Absolute Positive Change (APC) and Absolute Negative Change (ANC) are each ECG lead's absolute QRS amplitude change above and below the isoelectric baseline, respectively. Total Amplitude Change (TAC) is the sum of APC and ANC within each ECG lead. Absolute Amplitude Change (AAC) is the combined sum of TACs from the selected ECG leads of the frontal (aVR, aVL, aVF) ECG plane. Percent Amplitude Change (PAC) is the percent ratio of AAC to BA.

FIG. 21 is a flowchart illustrating the horizontal PAC (%) calculation. Horizontal PAC is composed of measured QRS waveform amplitudes (μV) derived from selected ECG leads within the horizontal ECG plane. LeadX denotes the individual selected ECG leads within the horizontal (V1, V4, V6) ECG plane. Positive Amplitude (PA) is the sum of measured QRS waveform amplitudes above the isoelectric baseline (r/R and r′/R′) in each single ECG lead. Negative Amplitude (NA) is the sum of measured QRS waveform amplitudes below the isoelectric baseline (q/QS, s/S, and s′/S′) in each single ECG lead. Total Baseline Amplitude (TBA) is the sum of PA and NA within each ECG lead of the baseline ECG. Baseline Amplitude (BA) is the summation of TBAs from the selected ECG leads in the horizontal (V1, V4, V6) ECG plane. Absolute Positive Change (APC) and Absolute Negative Change (ANC) are each ECG lead's absolute QRS amplitude change above and below the isoelectric baseline, respectively. Total Amplitude Change (TAC) is the sum of APC and ANC within each ECG lead. Absolute Amplitude Change (AAC) is the combined sum of TACs from the selected ECG leads of the horizontal (V1, V4, V6) ECG plane. Percent Amplitude Change (PAC) is the percent ratio of AAC to BA.

FIG. 22 is a flowchart illustrating the frontal PTVAC (%) calculation. Frontal PTVAC (%) is composed of measured QRS waveform time-voltage areas (μV·ms) derived from selected ECG leads within the frontal plane. Leadx denotes individual ECG leads within the frontal (aVR, aVL, aVF) ECG plane. Positive Area (PA) is the sum of measured QRS waveform time-voltage areas above the isoelectric baseline (r/R and r′/R′) in each single ECG lead. Negative Area (NA) is the sum of measured QRS waveform time-voltage areas below the isoelectric baseline (q/QS, s/S, and s′/S′) in each single ECG lead. Total Baseline Time-Voltage Area (TBTVA) is the sum of PA and NA within the selected individual ECG leads of the baseline ECG. Baseline Time-Voltage Area (BTVA) is the summation of TBTVAs from the selected ECG leads in the frontal (aVR, aVL, aVF) ECG plane. Absolute Positive Change (APC) and Absolute Negative Change (ANC) are each ECG lead's absolute QRS time-voltage area change above and below the isoelectric baseline, respectively. Total Time-Voltage Area Change (TTVAC) is the sum of APC and ANC within an individual ECG lead. Absolute Time-Voltage Area Change (ATVAC) is the combined sum of TTVACs from the selected ECG leads of the frontal (aVR, aVL, aVF) ECG planes. Percent Time-Voltage Area Change (PTVAC) is the percent ratio of ATVAC to BTVA.

FIG. 23 is a flowchart illustrating the horizontal PTVAC (%) calculation. Horizontal PTVAC (%) is composed of measured QRS waveform time-voltage areas (μV·ms) derived from selected ECG leads within the horizontal plane. Leadx denotes the selected individual ECG leads within the horizontal (V1, V4, V6) ECG plane. Positive Area (PA) is the sum of measured QRS waveform time-voltage areas above the isoelectric baseline (r/R and r′/R′) in each selected ECG lead. Negative Area (NA) is the sum of measured QRS waveform time-voltage areas below the isoelectric baseline (q/QS, s/S, and s′/S′) in each selected ECG lead. Total Baseline Time-Voltage Area (TBTVA) is the sum of PA and NA within the selected ECG leads of the baseline ECG. Baseline Time-Voltage Area (BTVA) is the summation of TBTVAs from the selected ECG leads in the horizontal (V1, V4, V6) ECG plane. Absolute Positive Change (APC) and Absolute Negative Change (ANC) are each selected ECG lead's absolute QRS time-voltage area change above and below the isoelectric baseline, respectively. Total Time-Voltage Area Change (TTVAC) is the sum of APC and ANC within each selected ECG lead. Absolute Time-Voltage Area Change (ATVAC) is the combined sum of TTVACs from the selected ECG leads of the horizontal (V1, V4, V6) ECG plane. Percent Time-Voltage Area Change (PTVAC) is the percent ratio of ATVAC to BTVA.

FIG. 24 is a flow chart describing the disclosed automated method of classifying wide complex tachycardias (WCTS) from ECG data in one aspect.

FIG. 25A is a schematic diagram describing the calculation of TVA for multiphasic waveforms.

FIG. 25B is a schematic diagram describing the calculation of TVA for monophasic waveforms.

FIG. 26 contains graphs of the receiver operating characteristic curves for a logistic regression model developed using only percent monophasic TVA to differentiate WCTs.

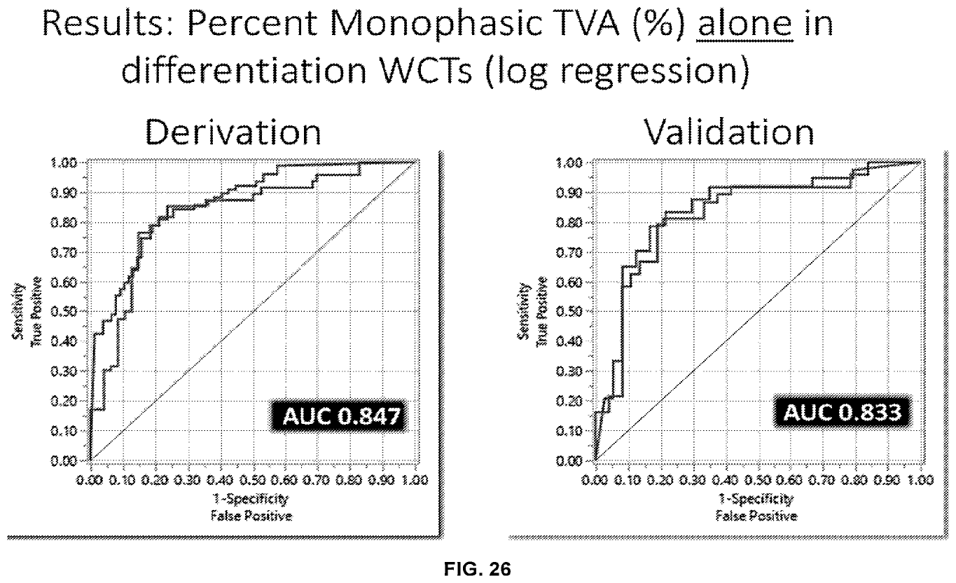

FIG. 27 contains graphs of the receiver operating characteristic curves for a logistic regression model developed using percent monophasic TVA, frontal PAC, horizontal PAC, and WCT QRS duration to differentiate WCTs.

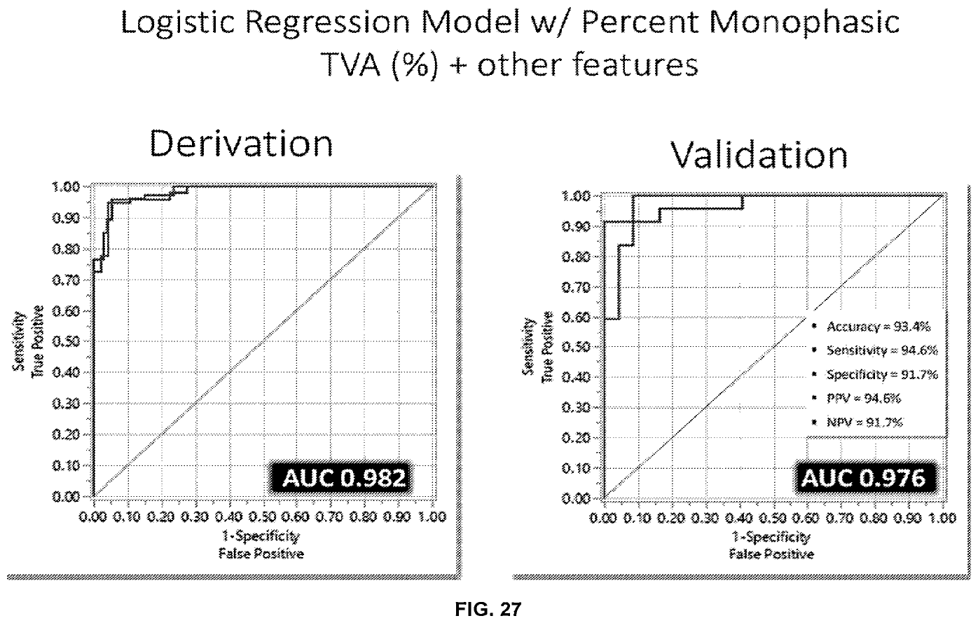

FIG. 28 contains histograms of the frequency of SWCTs (upper plot) and VTs (lower plot) identified using the model of FIG. 26.

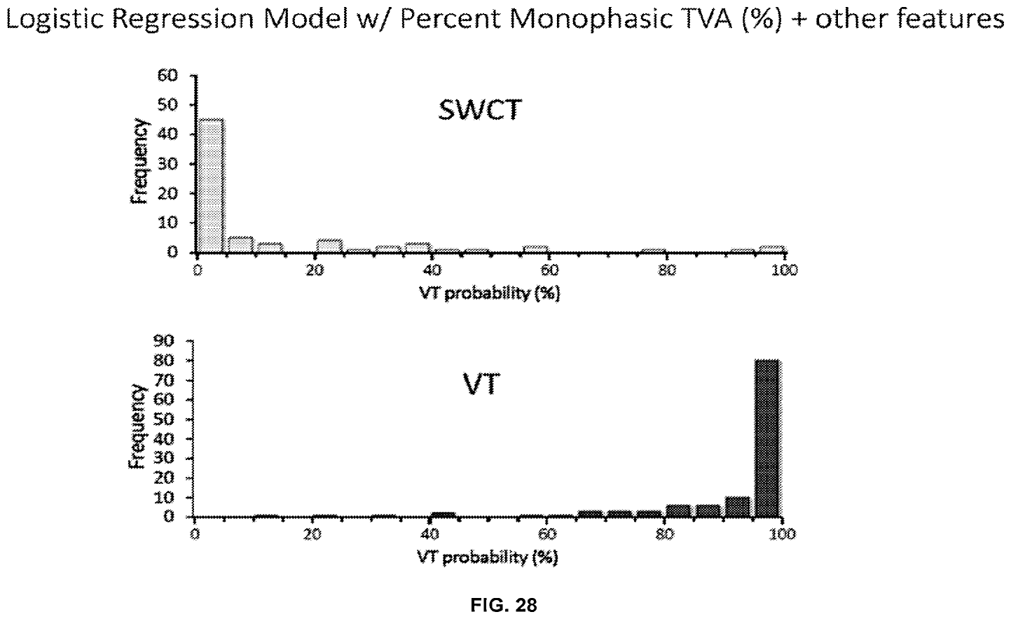

FIG. 29 contains graphs of the receiver operating characteristic curves for a support vector machine (SVM) model developed using percent monophasic TVA, frontal PAC, horizontal PAC, and WCT QRS duration to differentiate WCTs.

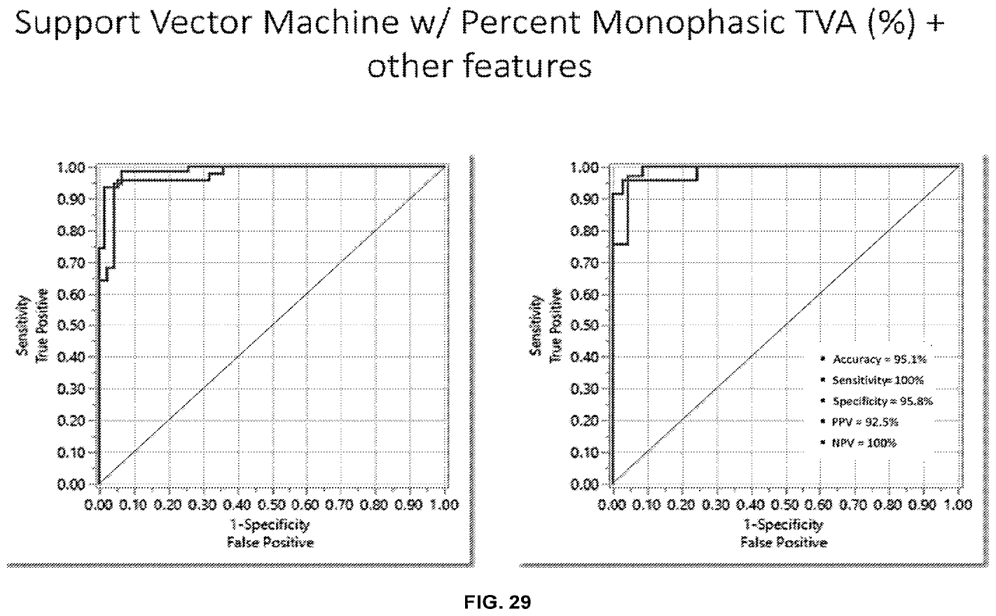

FIG. 30 contains graphs of the receiver operating characteristic curves for a random forest (RF) model developed using percent monophasic TVA, frontal PAC, horizontal PAC, and WCT QRS duration to differentiate WCTs.

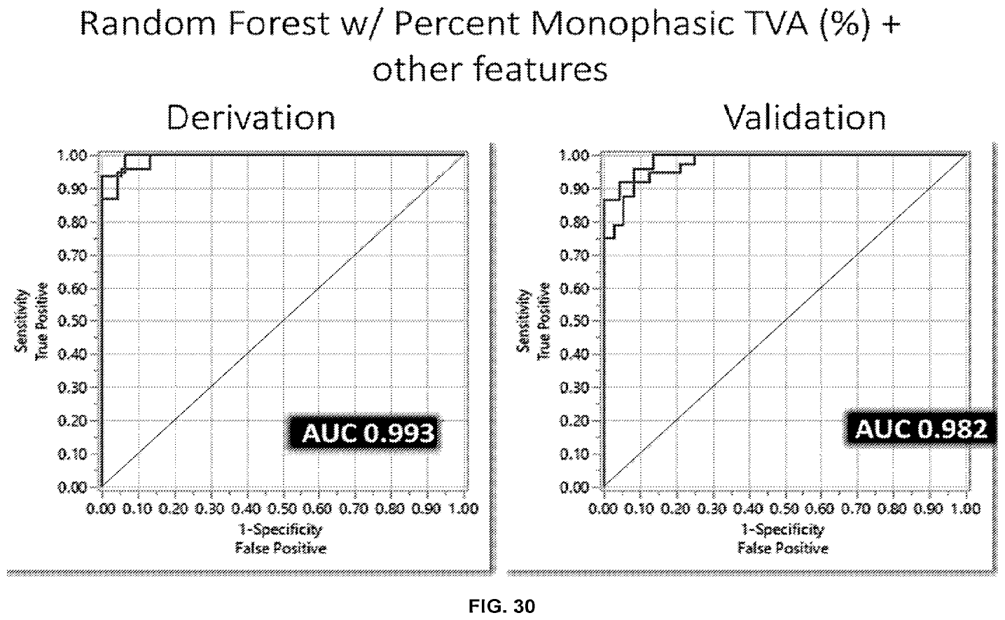

FIG. 31 contains graphs of the receiver operating characteristic curves for an artificial neural network (ANN) model developed using percent monophasic TVA, frontal PAC, horizontal PAC, and WCT QRS duration to differentiate WCTs.

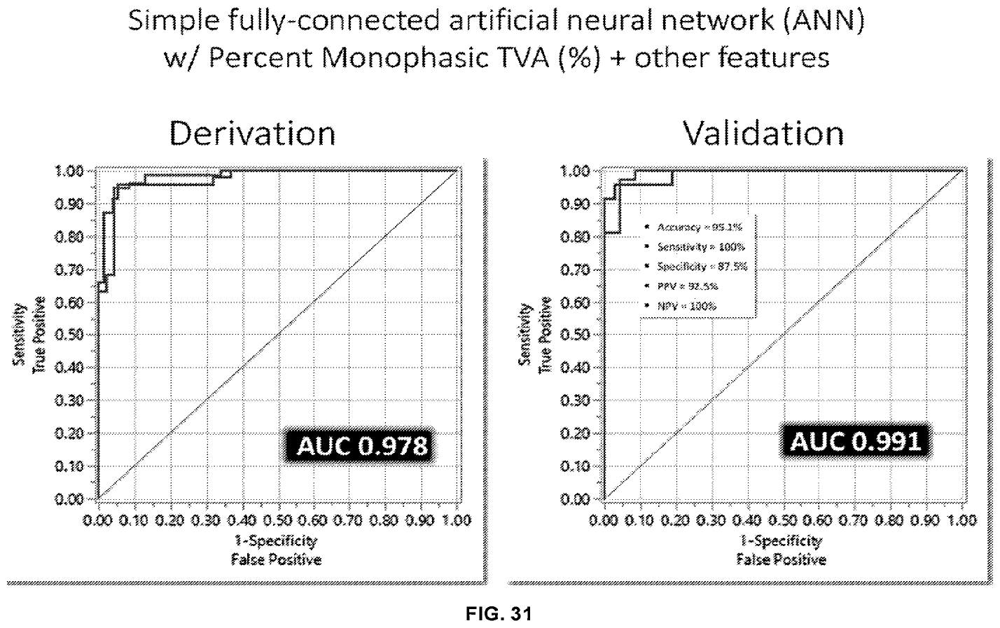

FIG. 32 is a schematic diagram showing an exemplary 12-lead ECG recording illustrating the readouts and corresponding QRS complexes of each ECG lead.

FIG. 33 is a flowchart illustrating the steps of the disclosed method of WCT ECG differentiation in one aspect.

### DETAILED DESCRIPTION OF THE INVENTION

The systems and methods of the present disclosure are based on the discovery that accurate and automatic WCT differentiation may be accomplished through the use of engineered features derived from computerized data of (i) WCT ECG alone and (ii) paired WCT and baseline ECGs. Once automated, the disclosed WCT differentiation models, which incorporate predictive engineered features derived from computerized ECG data, are suitable for integration into commercially available ECG interpretation software platforms to help clinicians distinguish VT and SWCT accurately and quickly select appropriate treatment.

In various aspects, systems and methods for wide QRS complex tachycardia (WCT) differentiation into ventricular tachycardia (VT) and supraventricular wide QRS tachycardia (SWCT) suitable for use with patients with and without a baseline ECG are disclosed herein. The disclosed systems and methods make use of at least several existing engineered features disclosed herein as well as two additional engineered features disclosed herein: percent monophasic time-voltage area [PMonoTVA] [%] and percent monophasic amplitude PMonoAmp [%]. In some aspects, the engineered features are derived from computerized ECG measurements as described herein. In various aspects, the disclosed engineered features provide for the differentiation of WCTs based on the WCT ECG alone without the need for a comparison baseline ECG. In various additional aspects, the engineered features described herein are suitable for incorporation into automated WCT differentiation models applicable to patients who present with or without corresponding baseline ECGs.

In various aspects, the disclosed systems and methods provide an estimated likelihood (i.e., probability or odds) of VT or SWCT to medical providers based on the automated analysis of WCT ECG. This estimated likelihood serves as cognitively meaningful clinical data that could supplement or replace information obtained by a provider using traditional (i.e., manual) methods to differentiate WCTs including, but not limited to, the Brugada algorithm.

In various aspects, the disclosed WCT differentiation systems and methods overcome at least some of the shortcomings of existing manual and automated WCT differentiation systems. In some aspects, the disclosed systems and methods enhance the diagnostic capability of automated electrocardiogram (ECG) interpretation, a critical component that supports clinical decision-making by medical providers. In other aspects, the disclosed systems and methods provide prompt and accurate wide complex tachycardia (WCT) differentiation into ventricular tachycardia (VT) and supraventricular wide complex tachycardia (SWCT), which is an undeniably common, vital, and challenging clinical task. In other additional aspects, the disclosed systems and methods automatically differentiate wide complex tachycardias (WCTs) into ventricular tachycardia (VT) and supraventricular wide complex tachycardia (SWCT), thereby reducing the variability and potential error introduced by manual methods and freeing up the practitioners to attend to the treatment of their patients.

In various aspects, the disclosed systems and methods transform readily available computerized ECG data into engineered features as described in additional detail herein. Without being limited to any particular theory, these engineered features capture and quantify cognizable underlying electrophysiology principles, namely that SWCT and VT are characterized by fundamental differences in the extent and efficiency to which they utilize the heart's native conduction system. In various aspects, these engineered features are suitable for analysis using machine learning algorithms or other automated classification models to accomplish automatic and accurate VT and SWCT classification using computerized ECG interpretation software including, but not limited to, MUSE software (GE Healthcare; Milwaukee, WI).

In various aspects, any suitable machine learning algorithms or other automated classification models may be used by the disclosed systems and methods to differentiate WCTs without limitation. Non-limiting examples of suitable machine learning algorithms or other automated classification models include logistic regression, artificial neural networks, random forest models, support vector machines, and any other suitable ML or other automated classification algorithms.

As disclosed in the Examples below, automated WCT differentiation models capable of accurately distinguishing VT and SWCT based on WCT ECG data were developed and evaluated. Comparable diagnostic performances among the various WCT differentiation model subtypes were generally demonstrated when applied to a broad collection of WCT differentiation engineered features. In addition, two specific WCT differentiation models were developed using logistic regression methods and evaluated for accuracy, sensitivity, and AUC. The first model, WCT Model I, was implemented using the WCT ECG data alone, and the second model, WCT Model II, required data from both the WCT and baseline ECG. In general, the WCT Model II demonstrated superior overall accuracy, sensitivity, and AUC compared to WCT Model I. However, the two models performed similarly in the case of diagnostic specificity for VT.

### WCT Differentiation Methods

In various aspects, methods of identifying ventricular tachycardia (VT) or supraventricular wide QRS tachycardia (SWCT) in a patient with wide QRS complex tachycardia (WCT) based on the patient's electrocardiogram (ECG) readings are disclosed herein. In some aspects, the ECG readings are WCT ECG readings obtained from the patient during wide QRS complex tachycardia (WCT). In other aspects, the ECG readings further include baseline ECG readings obtained from the patient outside of the WCT event in addition to the WCT ECG readings.

In various aspects, the disclosed methods transform WCT ECG readings, or alternatively WCT ECG readings in combination with baseline ECG readings into a classification of the WCT as VT or SWCT and/or a probability that the WCT is a VT or SWCT. As described herein, the information obtained using the disclosed method may be used by a practitioner to inform the selection of an appropriate treatment strategy.

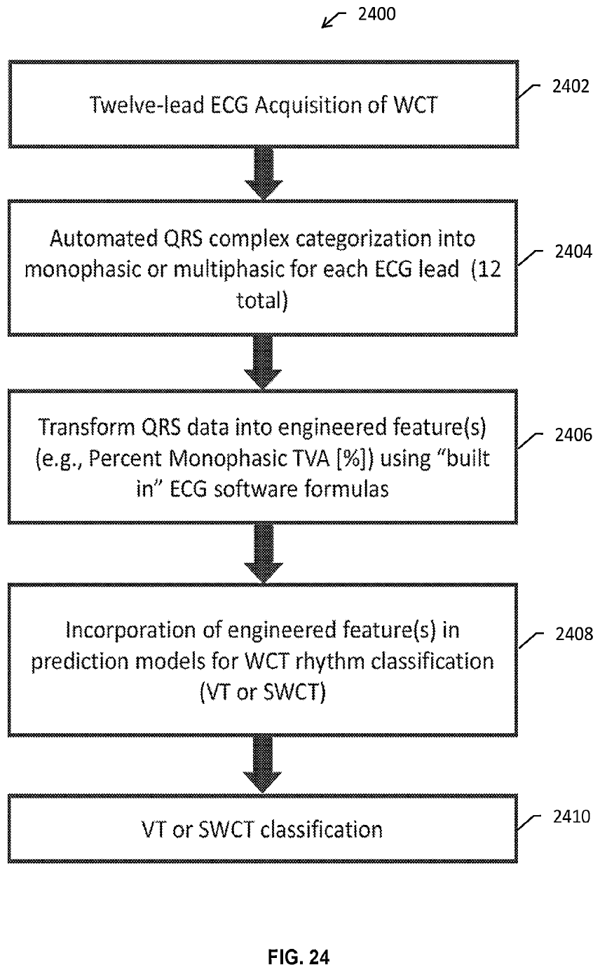

The steps of a method 2400 of developing a prediction model to be used by WCT differentiation methods in various aspects described herein are illustrated in FIG. 24. The method 2400 includes receiving 12-lead WCT ECG data at 2402 and automatically categorizing the QRS complex as either monophasic or multiphasic for the ECG data from each of the twelve leads at 2404. The QRS data is then transformed into one or more of the engineered features at 2406, and one or more of these engineered features are incorporated into one of the predictive models described herein at 2408. The predicted WCT rhythm classification determined at 2408 is compared to a previously-determined classification at 2410 to assess the accuracy of the predictive model. In some aspects, the method 2400 is performed iteratively for a population of patients, and the predictive model may be refined iteratively at 2408 and 2410 by evaluating predictive models produced using different combinations of engineered features determined at 2406.

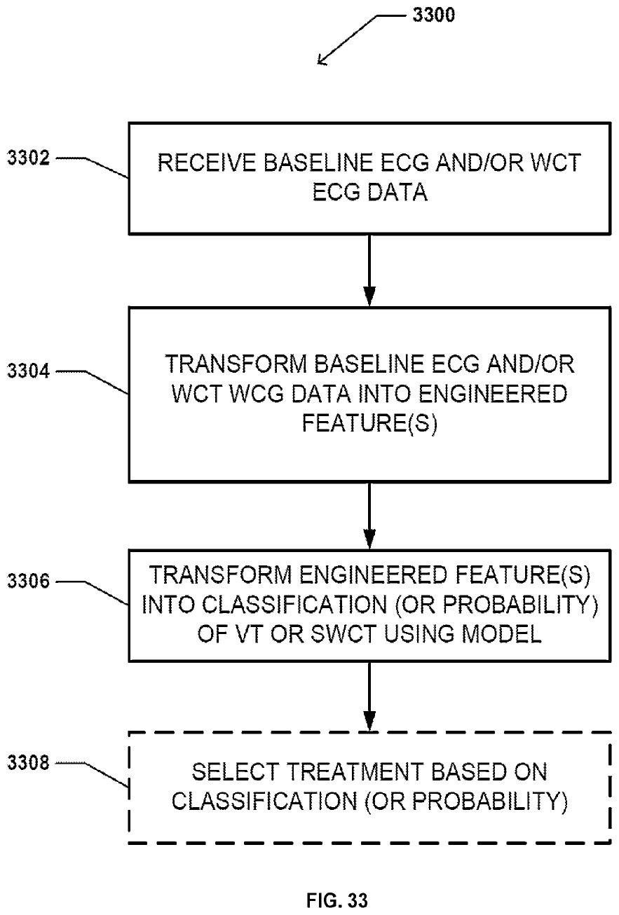

FIG. 33 is a flowchart illustrating the main steps of the disclosed WCT differentiation method 3300 in one aspect. The method 3300 includes receiving baseline ECG data and/or WCT ECG data at 3302, transforming the baseline ECG data and/or WCT ECG data into engineered features at 3304 using predetermined relationships at 3306, and transforming the engineered features into a classification or estimated probability of VT or SWCT using a model at 3306. In some aspects, the method further includes selecting a treatment at 3308 based on the classification or probability of VT or WCT obtained at 3306. In some aspects, the method accomplishes the WCT differentiation at 3306 based solely on the WCT ECG data alone. In other aspects, the method accomplishes the WCT differentiation at 3306 based on the WCT ECG data as well as baseline ECG data.

### ECG Data

In some aspects, the WCT ECG and/or baseline ECG data comprise the voltage readings obtained by the ECG system or device as described herein. In other aspects, the WCT ECG and/or baseline ECG data further comprise at least one summary parameter including, but not limited to, the durations and/or amplitudes characterizing a QRS complex waveform as described herein. In other aspects, where baseline SCG is available, the WCT ECG and baseline ECG data further comprise at least one comparison parameter including, but not limited to, differences between corresponding summary parameters obtained from the WCT ECG data and the baseline ECG data, respectively.

In various aspects, the ECG data is measured using any suitable ECG measurement system or device without limitation. Non-limiting examples of suitable ECG devices include a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automatic implantable cardioverter defibrillator (AICD), an automated external defibrillator (AED), and any other suitable ECG measurement system or device.

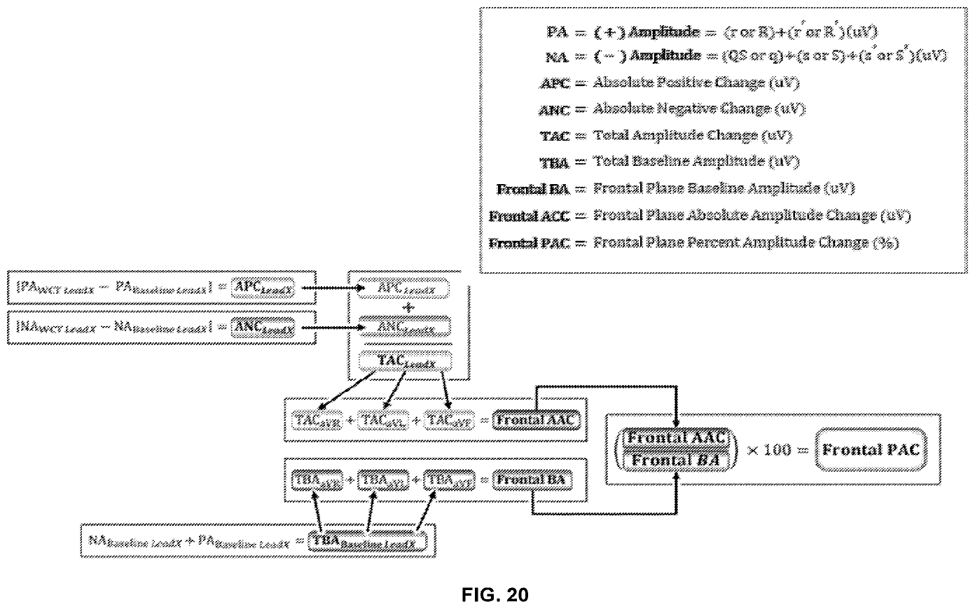

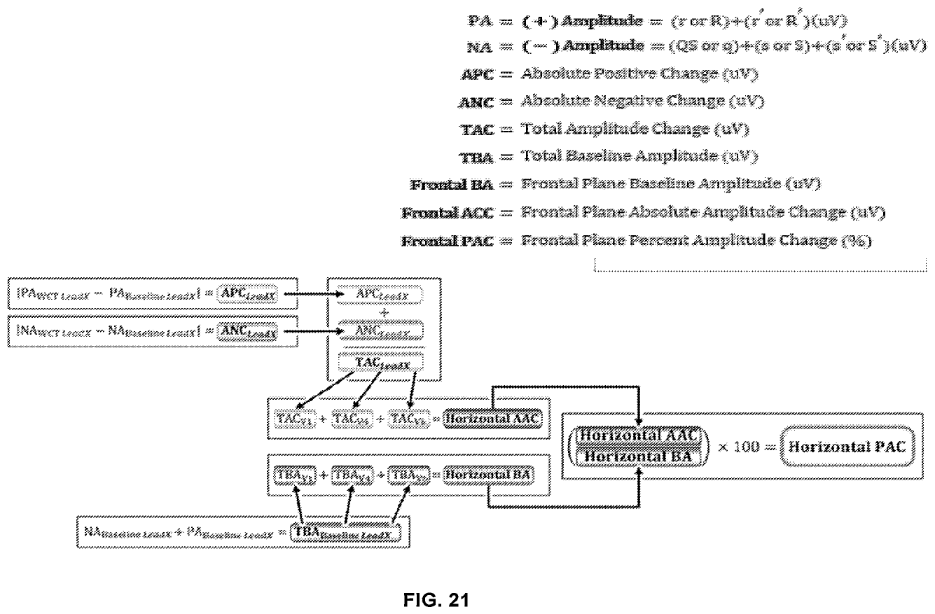

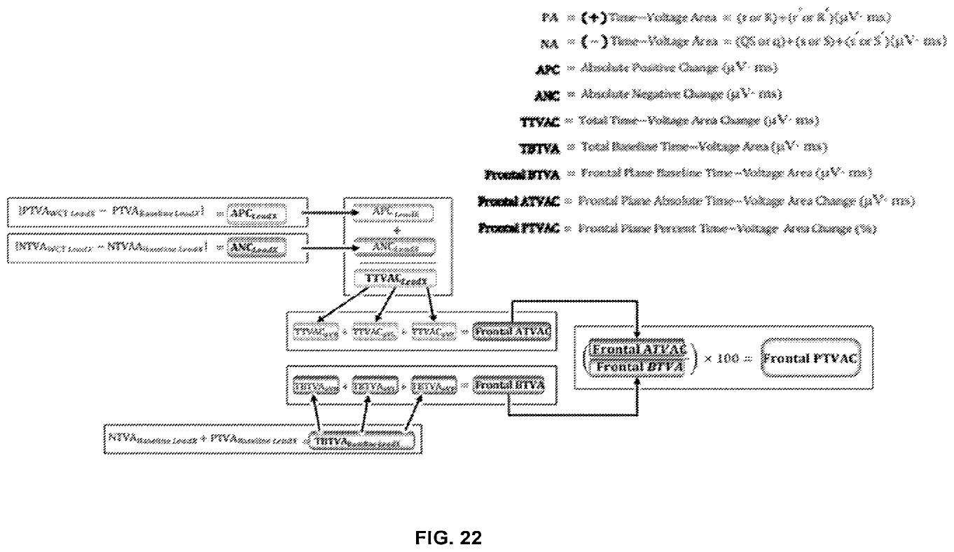

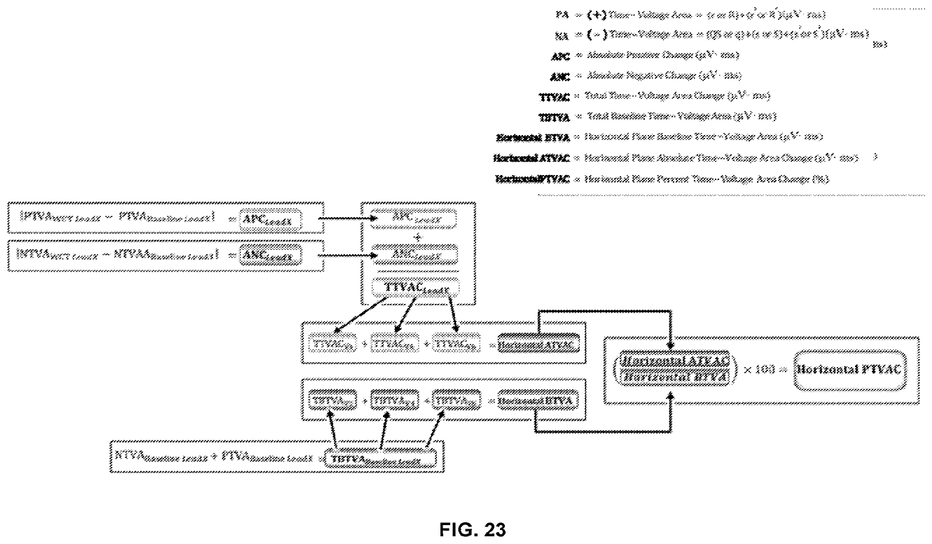

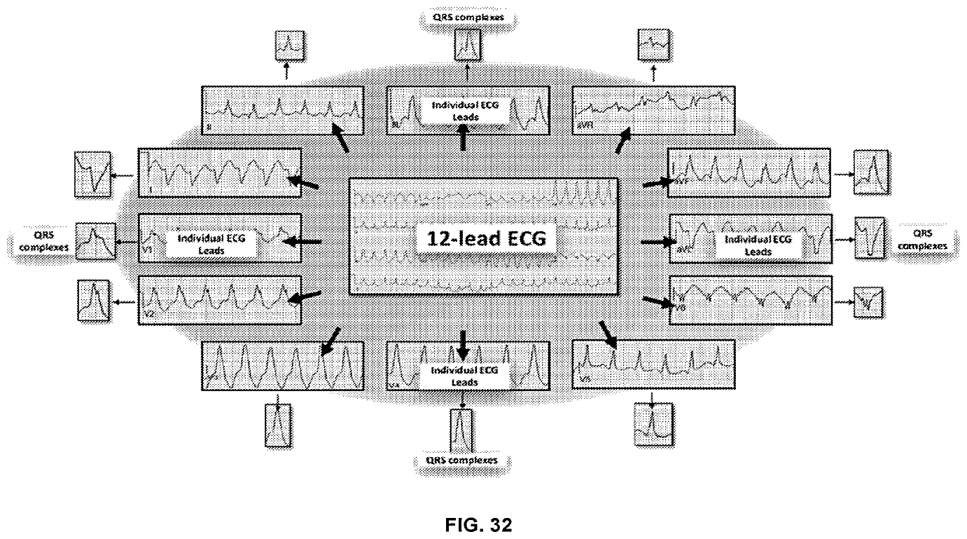

In one aspect, the ECG data is measured using a 12-lead ECG device. FIG. 32 is a schematic illustration showing exemplary measurements obtained using a 12-lead ECG device. As illustrated in FIG. 32, the 12 ECG leads are denoted according to their positioning on the patient as V1, V2, V3, V4, V5, V6, I, aVL, II, aVF, III, and aVR. Each of the 12 leads records a unique QRS complex representative of the heart's ventricular depolarization, and the ECG recordings from the ECG leads or various combinations thereof may be analyzed using the disclosed method as described herein. As described in further detail herein, the voltage amplitude measurements from specific leads including, but not limited to, frontal ECG plane leads V1, V4, V6 and horizontal ECG plane leads aVL, aVF, aVR are used in the formulas to generate engineered features including, but not limited to, frontal PACs (see FIG. 20) and horizontal PACs (FIG. 21) in some aspects. In other additional aspects, frontal PTVAC (%) (FIG. 22), and horizontal PTVAC (%) (FIG. 23) are similarly generated.

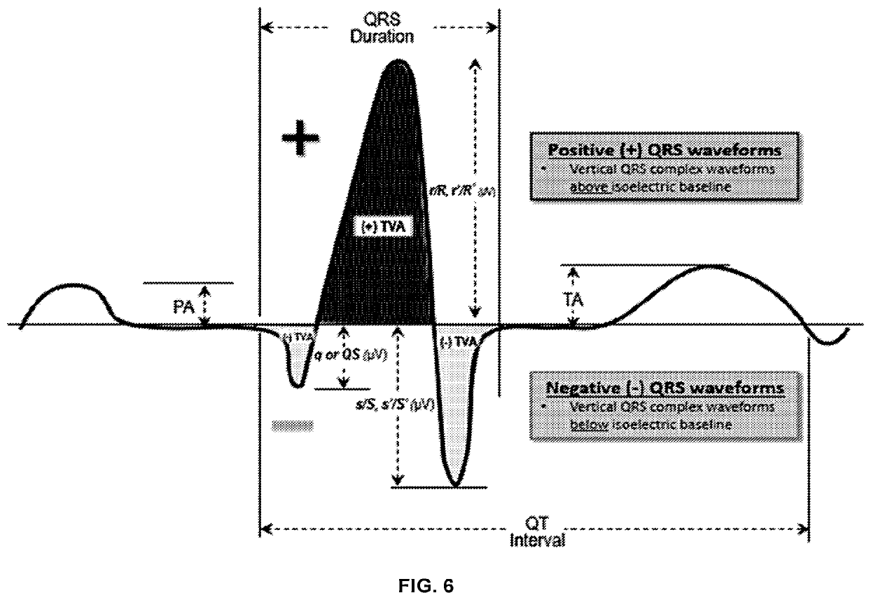

FIG. 6 is a schematic representation of a stereotypical ECG pattern for a single heartbeat. The QRS complex waveform is a combination of three graphical deflections: the Q wave having a downward deflection immediately following the P wave; the R wave having an upward deflection immediately following the Q wave; and the S wave having a downward deflection following the R wave. The Q wave, R wave, and S wave occur in rapid succession in a QRS complex waveform that occurs over a time interval denoted as QRS duration. A T wave follows the S wave. Each wave has amplitude denoted as PA, QA, RA, SA, and TA. In addition, the QT interval is the time interval extending from the onset of the QRS complex waveform to the end of the T wave. The QRS complex is divided into positive (+) amplitudes and negative (−) amplitudes. The positive (+) amplitudes are the vertical QRS complex deflections above the isoelectric baseline, namely the amplitudes of the r/R wave and r′/R′ wave. The negative (−) amplitudes are the vertical QRS complex deflections below the isoelectric baseline, namely the amplitudes of the q wave or QS wave, the s/S wave, and the s′/S′ wave.

Referring again to FIG. 6, the QRS complex is divided into positive (+) time-voltage areas (TVA) and negative (−) TVAs, marked as blue and yellow regions, respectively. The positive (+) TVAs are the TVAs of the vertical QRS complex deflections above the isoelectric baseline, namely the TVAs of the r/R wave (and r′/R′, not shown). The negative (−) TVAs are the TVAs of the vertical QRS complex deflections below the isoelectric baseline, namely the TVAs of q (or QS, not shown) and s/S (and s′/S′, not shown) waves. Computerized ECG interpretation software, such as the MUSE software provided by GE Healthcare, automatically measures QRS complex waveform attributes, namely q or QS, r/R, s/S, r′/R′, s′/S′ durations (ms), amplitudes (μV), and time-voltage areas (μV·ms).

In some aspects, QRS complex waveform attributes, including, but not limited to, q or QS, r/R, s/S, r′/R′, s′/S′ durations (ms), amplitudes (μV), and time-voltage areas (μV·ms) are provided by automated data analysis software including, but not limited to, GE Healthcare's MUSE ECG interpretation software and databank. By way of non-limiting example, automated data analysis software may provide the amplitudes (_A), durations (_D), and voltage areas (_VA) for the various waves including, but not limited to, values denoted as PA, PPA, QA, QD, RA, RD, SA, SD, RPA, RPD, SPA, and SPD. In various aspects, any other computerized ECG interpretation software can be used to derive these QRS complex waveform attributes without limitation. It is to be noted that the measurements described herein are not routinely shown on the ECG paper recordings, but are available within the ECG interpretation software databanks.

In some aspects, if baseline ECG data are available for a patient, the baseline ECG data may be compared to the WCT WCG data to derive additional comparison parameters indicative of variations in the heart's ventricular depolarization relative to a normal ventricular depolarization. In various aspects, the comparison parameters include any changes in any of the QRS waveform parameters relative to corresponding baseline QRS waveform parameters as described herein without limitation. In some aspects, these comparison parameters are produced using the automated data analysis software described herein. By way of non-limiting example, automated data analysis software may produce a variety of comparison parameters including, but not limited to differences in QRS duration (ms), changes in frontal plane R wave axis (°), and changes in frontal plane T wave axis (°) between the WCT and baseline ECG data.

### Engineered Features

Referring again to FIG. 33, the method 3300 further includes transforming the baseline and/or WCT ECG data into engineered features at 3304. In various aspects, the engineered features comprise various combinations of subsets of the waveform attributes described herein. In various aspects, the subsets of waveform attributes comprise corresponding waveform attributes derived from the voltage measurements obtained by at least two different leads of the baseline and/or WCT ECG data.

In various aspects, the engineered features are provided in the form of parameter values produced according to a predetermined relationship. Any suitable predetermined relationship may be used to produce the parameter values without limitation including, but not limited to, a data analysis method, an algorithm, or an equation. Non-limiting examples of engineered features suitable for use in the methods disclosed herein are described in PCT International Publication No. WO/2020/01471, the content of which is incorporated by reference herein in its entirety.

### Percent Monophasic Time Voltage Area and Amplitude

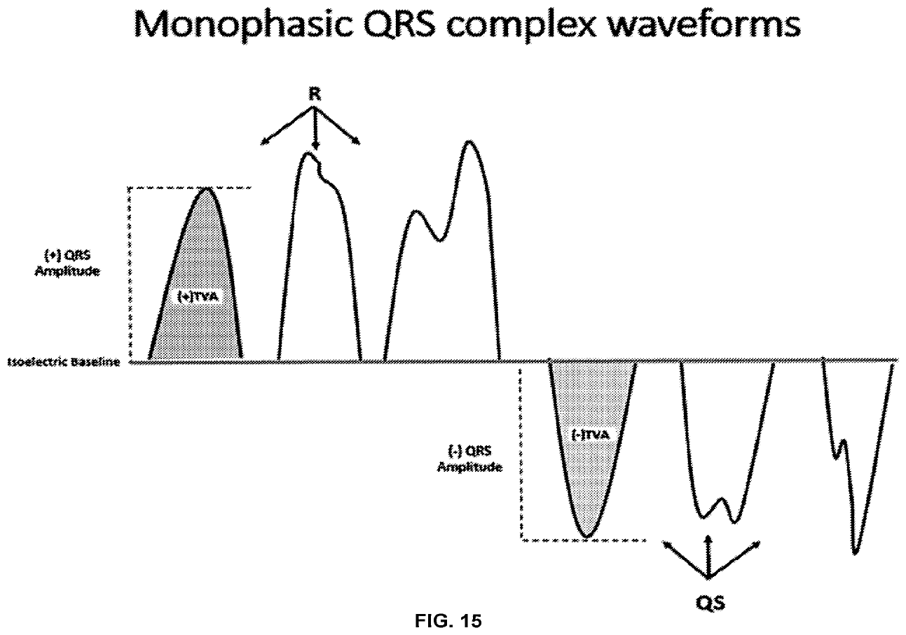

In various aspects, the engineered features include PMonoTVA which quantifies the proportion of the time-voltage areas (TVA) of the QRS waveforms that include monophasic QRS complexes (see FIG. 15) within the WCT ECG data. In some aspects, the TVA of the QRS waveforms that include monophasic QRS complexes may be calculated as illustrated in FGI. 25B. In other aspects, the engineered features further include PMonoA which quantifies the proportion of amplitudes of the QRS waveforms that include monophasic QRS complexes within the WCT ECG data.

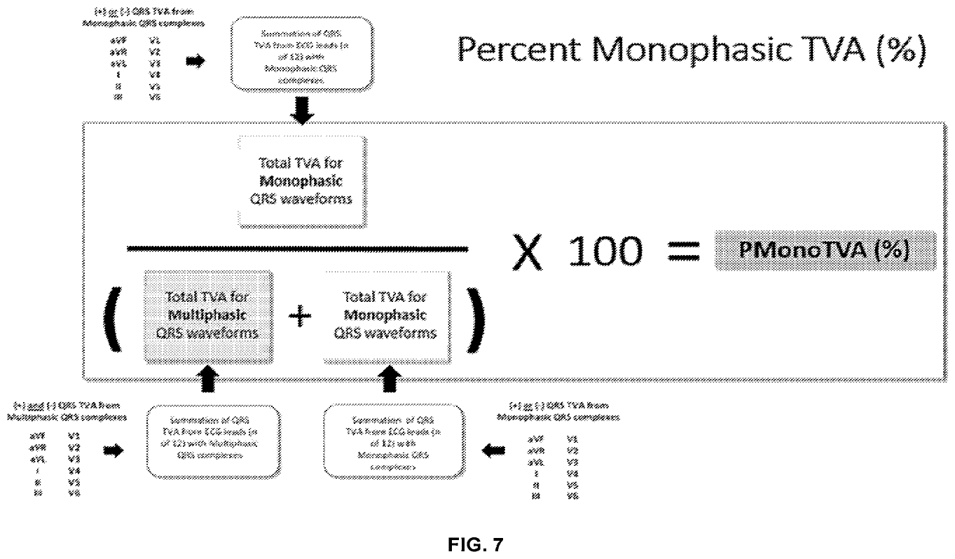

Percent monophasic time-voltage area (PMonoTVA), as used herein, refers to the percentage of QRS TVA contained by monophasic QRS complexes of the 12-lead ECG. In various aspects, PMonoTVA is calculated as the percentage ratio of the sum of QRS TVA from ECG leads with monophasic QRS complexes (i.e., monophasic TVA) to the entire sum of QRS TVA from all ECG leads (i.e., monophasic TVA plus multiphasic TVA), as illustrated schematically in FIG. 7. The TVA measurements used in the calculation of PMonoTVA are obtained from QRS complex waveforms (q/QS, r/R, s/S, r′/R′, and s′/S′) of the dominant QRS complex template of all individual leads of the 12-lead ECG (i.e., leads I, II, III, aVF, aVL, aVR, V1, V2, V3, V4, V5, and V6).

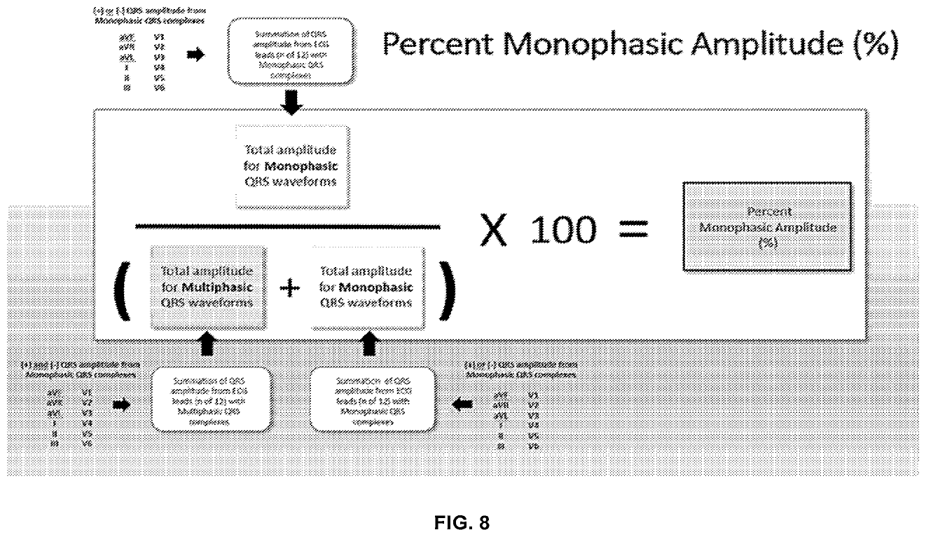

Percent monophasic amplitude (PMonoAmp), as used herein, refers to the percentage of QRS amplitude found on monophasic QRS complexes of the 12-lead ECG. PMonoAmp is calculated as the percentage ratio of the sum of QRS amplitude from ECG leads with monophasic QRS complexes (i.e., monophasic amplitude) to the entire sum of QRS amplitude from all ECG leads (i.e., monophasic amplitude plus multiphasic amplitude, as illustrated schematically in FIG. 8. Representative amplitude measurements are attained from QRS complex waveforms (q/QS, r/R, s/S, r′/R′, and s′/S′) of the dominant QRS complex template of individual leads of the 12-lead ECG (i.e., leads I, II, III, aVF, aVL, aVR, V1, V2, V3, V4, V5, and V6).

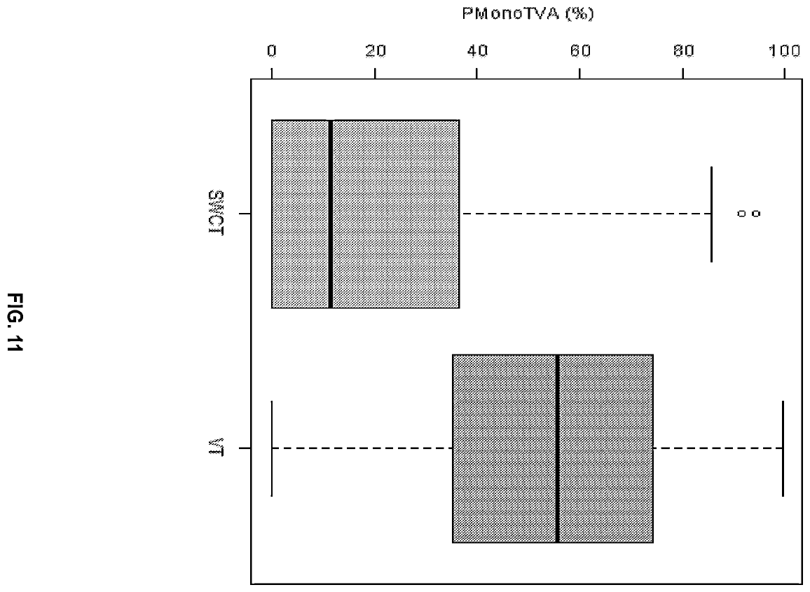

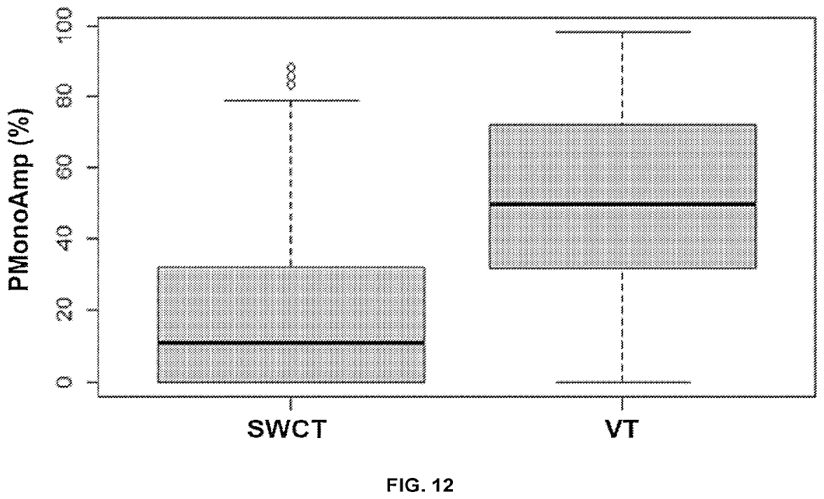

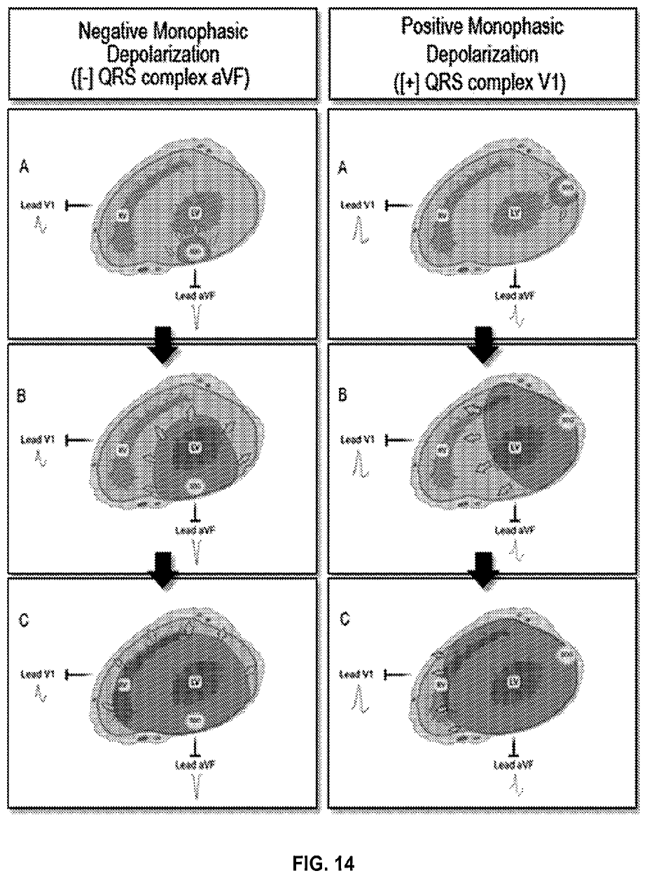

As discussed in the Examples below, higher PMonoTVA and PMonoAmp values occurred among patients with VT as compared to patients with SWCT (see FIGS. 11 and 12, respectively). The conceptual basis for these engineered features helping differentiate WCTs relates to the manner in which ventricular depolarization most commonly occurs for VT and SWCT. Without being limited to any particular theory, for many patients with VT, ventricular depolarization wavefronts primarily spread from their site-of-origin (SOO) using cardiomyocyte-to-cardiomyocyte conduction, with little-to-no assistance from the specialized conduction tissue, as illustrated in FIG. 14. As a result, the ventricular depolarization wavefronts of VT tend to spread uniformly sans swift engagement and subdivision along an arborized His-Purkinje network that accounts for diametrically opposed and temporally separated QRS complex waveforms.

Correspondingly, patients with VT will often exhibit monophasic QRS complexes (i.e., monophasic ‘QS complexes’ for ventricular depolarization wavefronts propagating away from any of the 12 ECG leads or monophasic ‘R complexes’ propagating toward any of the 12 ECG leads), especially if the SOO is remote from specialized conduction tissue or subjacent to the ventricular epicardium. Exemplary monophasic QRS waveforms are shown illustrated schematically in FIG. 15. It is to be noted that monophasic QRS complex waveforms may possess positive or negative amplitudes with respect to the isoelectric baseline.

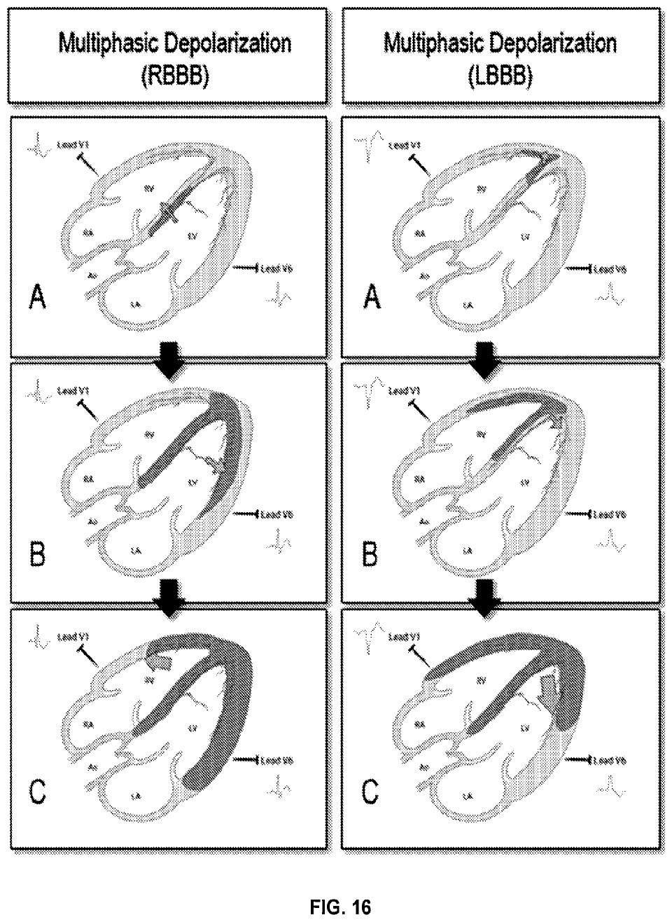

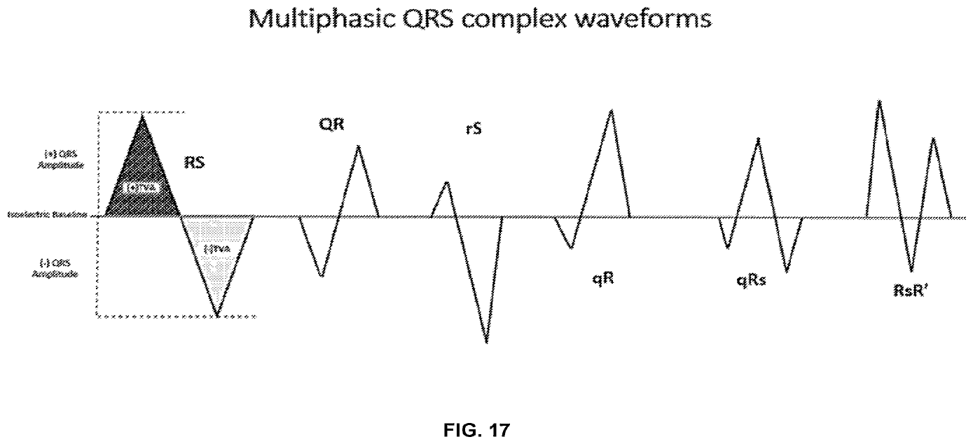

In contrast, most SWCT subtypes of WCT rely heavily upon the heart's specialized conduction tissue—not only as the primary link that couples supraventricular impulses to ventricular depolarization but also as the indispensable conduit by which biventricular depolarization is accomplished. Among SWCTs arising from aberrant conduction (e.g., left bundle branch block [LBBB] or right bundle branch block [RBBB]), the rapid initial stages of ventricular depolarization affected by via specialized conduction tissue conduction are subsequently followed by a slower ventricular depolarization wavefront, which relies upon slower cardiomyocyte-to-cardiomyocyte conduction, as illustrated schematically in FIG. 16. As a result, the separate and distinct components of ventricular depolarization (i.e., initial vs. late) tend to produce multiphasic QRS complexes having diametrically opposed and temporally separated QRS complex waveform components (i.e., positive [+] QRS waveforms [r/R, r′/R′] vs. negative [−] QRS waveforms [q/Q, s/S, s′/S′]), shown illustrated schematically in FIG. 17.

### Frontal/Horizontal Percent Amplitude and Time-Voltage Area Changes

In some aspects, the engineered features further include at least one of frontal percent amplitude change (Frontal PAC), horizontal percent amplitude change (Horizontal PAC), frontal percent time-voltage area change (Frontal PTVAC), and horizontal percent time-voltage area change (Horizontal PTVAC). Frontal and horizontal PAC and PTVAC are calculations that broadly quantify the extent of QRS amplitude change and TVA (time-voltage area) change, respectively, between WCT and baseline ECGs.

Frontal and horizontal PACs are quantifiable measures of QRS amplitude changes between paired WCT and baseline ECG recordings. The processes of calculating the frontal and horizontal PACs are illustrated schematically in FIGS. 20 and 21, respectively. They are derived from computerized QRS waveform (q/QS, r/R, s/S, r′/R′, and s′/S′) amplitude (μV) measurements from corresponding ECG leads of the frontal (aVR, aVL, aVF) and horizontal (V1, V4, V6) ECG planes, respectively.

Frontal and horizontal PTVACs are quantifiable measures of the degree of change in QRS TVA (time-voltage area) between paired WCT and baseline ECGs, The processes of calculating the frontal and horizontal PTVACs are illustrated schematically in FIGS. 22 and 23, respectively. They are derived from QRS complex waveform (q/QS, r/R, s/S, r′/R′, and s′/S′) TVA measurements from specific ECG leads within the corresponding frontal (aVR, aVL, aVF) or horizontal (V1, V4, V6) ECG planes, respectively.

As discussed in the Examples below, WCTs exhibiting larger frontal and horizontal PAC and PTVAC were more likely VT while WCTs demonstrating smaller frontal and horizontal PAC and PTVAC were more likely SWCT. These findings are in agreement with published results that demonstrated that paired WCT and baseline ECGs having greater frontal and/or horizontal PAC and PTVAC values more commonly developed among patients with VT. Similar to the electrophysiological mechanisms underpinning QRS axis change, these observations may be explained by the differences by which VT and SWCT ordinarily depolarize the ventricular myocardium. In general, VT categorically demonstrates greater independence in the manner that ventricular depolarization may occur compared to its relatively constrained SWCT counterpart. Thus, the broad expanse of VT manifestations yields a vastly greater number of distinct QRS complexes than those that can develop from SWCT, which in turn helps enable larger QRS amplitude and TVA changes for VT compared to SWCT.

### WCT QRS Duration, Baseline QRS Duration, and QRS Duration Changes

In some aspects, the engineered features further include at least one of WCT QRS duration, Baseline QRS Duration, and QRS duration changes. QRS duration, as used herein, refers to the total duration of the QRS waveform, shown illustrated schematically in FIG. 6. WCT QRS duration and baseline QRS duration may be obtained from WCT and baseline ECG data, respectively using any suitable method without limitation including, but not limited to, manual ECG data analysis methods and automated ECG data analysis methods. In one aspect, WCT QRS duration and baseline QRS duration are obtained using any suitable automated data analysis software including, but not limited to, MUSE ECG interpretation software (GE Healthcare; Milwaukee, WI).

QRS duration change, as used herein, refers to the absolute difference in QRS duration (ms) measurements between paired WCT and baseline ECGs. In one aspect, QRS duration change is obtained by calculating the difference between the WCT QRS duration and the baseline QRS duration obtained using MUSE ECG interpretation software as described herein.

Without being limited to any particular theory, patients presenting with VT may exhibit greater WCT QRS duration, baseline QRS duration, and change in QRS duration than patients with SWCT. Previously published results have demonstrated that engineered features relating to QRS duration (i.e., WCT QRS duration, baseline QRS duration, and absolute change in QRS duration between the WCT and baseline ECG) are useful in differentiating WCTs. WCTs having greater WCT QRS duration, baseline QRS duration, and absolute change in QRS duration are more likely secondary to VT, while WCTs demonstrating lesser values were more likely due to SWCT.

Prior research has demonstrated that VT commonly expresses longer QRS durations than SWCT. Though VT and SWCT occupy broad and overlapping QRS duration ranges, the dissimilarity in QRS duration magnitude between these rhythms may be explained by differences in the manner by which VT and SWCT typically depolarize the ventricular myocardium. As noted herein, VT commonly relies upon a less efficient means of wavefront propagation (i.e., cardiomyocyte-to-cardiomyocyte) to depolarize the ventricular myocardium, which in turn tends to produce a prolonged QRS duration. In contrast, most SWCTs make use of the patient's native conduction system to rapidly accomplish the initial stages of ventricular depolarization, thereby reducing the amount of ventricular myocardium that must be depolarized by slower cardiomyocyte-to-cardiomyocyte conduction.

Previously published results suggest that the differences in baseline QRS duration between VT and SWCT patients may be explained by the underlying myocardial substrate from which both heart rhythms commonly develop. Given that VT, by and large, emerges from patients who have underlying structural heart disease, patients who develop VT will tend to have a higher prevalence of underlying ventricular conduction abnormalities (e.g., bundle branch block) or baseline ventricular pacing from an implanted intracardiac device (e.g., primary prevention ICD); both of which may be directly responsible for a longer baseline QRS duration. Furthermore, antiarrhythmic medications (e.g., sodium-channel blockers) known to cause baseline QRS duration prolongation are frequently provided to patients who have a history of VT.

The differences in the extent of QRS duration change upon WCT onset or offset between VT and SWCT are thought to be directly secondary to their differing means of ventricular depolarization. Given that VT commonly originates well outside the normal conduction system, it will often generate a large increase in QRS duration, especially among patients whose baseline heart rhythm exhibits rapid and efficient ventricular depolarization via a healthy His-Purkinje network. In contrast, many SWCTs appropriate the same conduction system pathways as the baseline heart rhythm and will express minimal changes to QRS duration. Only SWCTs due to ‘functional’ aberrancy or emerging preexcitation, which generate newfound ventricular depolarization delays, demonstrate large QRS duration changes between the WCT and baseline ECGs.

### QRS Axis Change and T Wave Axis Change

In some aspects, the engineered features further include at least one of QRS axis change and T wave axis change. QRS axis change, as used herein, refers to the absolute difference in orientation of the frontal plane QRS axis (°) between paired WCT and baseline ECGs. T wave axis change, as used herein, refers to the absolute difference in the orientation of the frontal plane T wave axis between paired WCT and baseline ECGs.

As described in the Examples below, WCTs exhibiting larger QRS axis and T axis changes compared to the baseline rhythm were more likely VT, while WCTs demonstrating smaller QRS axis and T axis changes were more likely SWCT. Previously published results support the concept that changes in the direction of the mean electrical vector of ventricular depolarization (i.e., QRS axis change) and/or repolarization (i.e., T axis change) are valuable in differentiating the underlying WCT rhythm (i.e., VT or SWCT)—i.e., large changes predict VT and smaller changes predict SWCT. Without being limited to any particular theory, the conceptual basis for these observations relates to the manner by which VT and SWCT rhythms depolarize and repolarize the ventricular myocardium. While the large majority of SWCTs must activate the ventricular myocardium from a supraventricular origin by way of conduction pathways offered by the His-Purkinje network, VT can originate and propagate from any ventricular location, and the direction of wavefront propagation that patients may demonstrate has unbounded possibilities. As a result, VT rhythms may express an extensive variety of ventricular depolarization (i.e., QRS complex) and repolarization (i.e., T wave) patterns, resulting in significantly changed mean electrical axis orientations (i.e., QRS axis and/or T wave axis) compared to the corresponding axis orientation of the baseline ECG.

### WCT Differentiation Models

Referring again to FIG. 33, the method 3300 further includes transforming the engineered features obtained at 3304 into a classification of the SWCT using a model at 3306 in various aspects. In various other aspects, the method 3300 further includes transforming the engineered features obtained at 3304 into an estimated probability that the WCT is a VT or an SWCT using a model at 3306. Any suitable model may be used to transform the engineered features without limitation. In some aspects, a machine learning model is used to transform the engineered features into a classification or probability of VT or SWCT. Non-limiting examples of suitable machine learning models include genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In one exemplary method, a logistic regression model is used to transform the engineered features into a classification or probability of VT or SWCT.

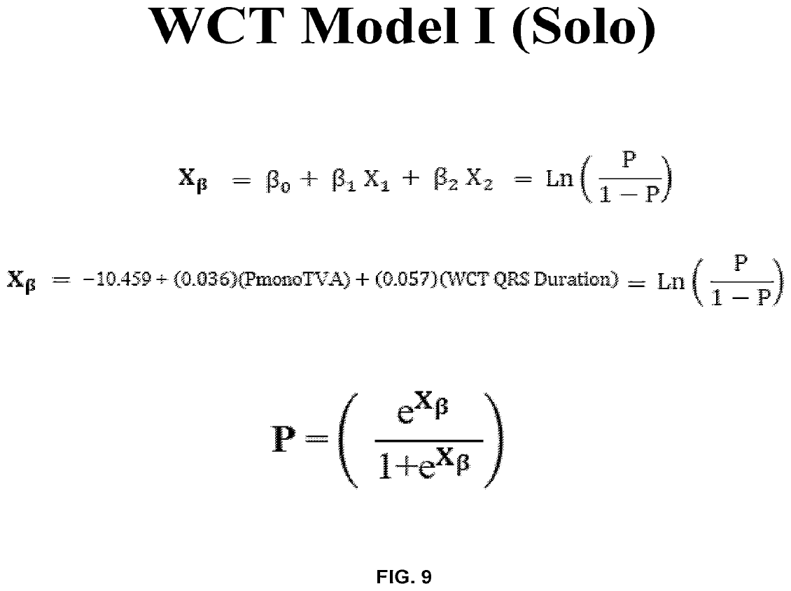

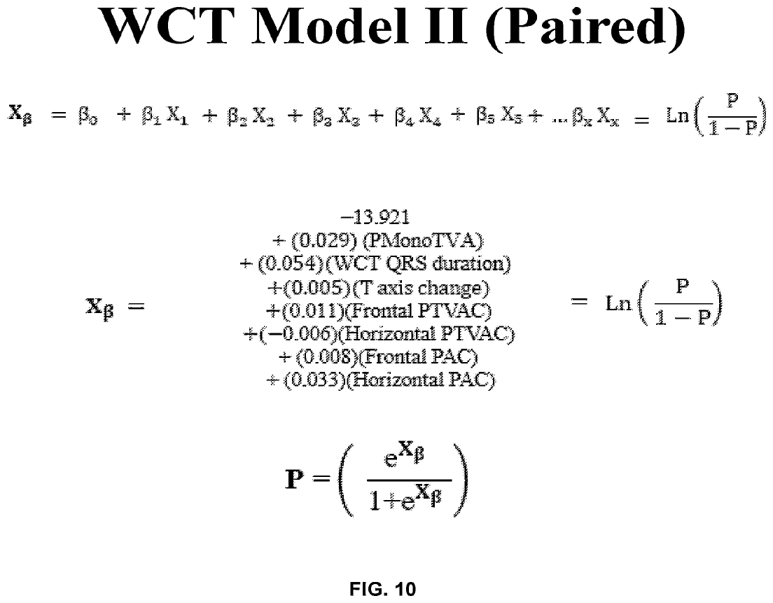

In various aspects, the engineered parameters derived from WCT ECGs or paired baseline and WCT ECGs are incorporated into one or more WCT formulas to calculate VT probability using a logistic regression model. In some aspects, logistic regression models including, but not limited to WCT Model I (see FIG. 9) make use of WCT predictor values derived solely from WCT ECG data. In other aspects, additional logistic regression models including, but not limited to WCT Model II (see FIG. 10) makes use of WCT predictor values derived solely from WCT ECG data as well as additional WCT predictor values derived from changes between baseline ECG data and WCT ECG data.

In various aspects, the machine learning model transforms engineering features derived from WCT ECG data only, without the need for baseline ECG data. The disclosed method in these aspects overcomes a universal limitation of existing automated WCT differentiation methods that required computerized data provided by both the WCT itself and its respective baseline ECG. In various aspects, the machine learning model is a binary outcome logistic regression model that transforms selected independent WCT predictors into a classification of VT or SWCT. Non-limiting examples of suitable independent WCT predictors include the engineered parameters PMonoTVA and WCT QRS Duration.

FIG. 9 is an illustration of a logistic regression model used to transform the selected engineered features into a probability P of VT. Each WCT predictor (Xx) is assigned beta coefficients (βx) according to their influence on the binary outcome (VT vs. non-VT). The “constant” term (B0) represents the y-intercept of the least-squares regression line. The discrete measured or calculated WCT predictor values are incorporated into a WCT formula including, but not limited to, the weighted sum formula as shown in FIG. 9.

By way of non-limiting example, WCT Model I makes use of two highly predictive engineered features derived solely from the WCT ECG: (i) a standard and universally reported ECG measurement (i.e., WCT QRS duration) and (ii) computationally-derived TVA measurements of QRS complex waveforms, PMonoTVA (see FIG. 7). The WCT Model I delivers accurate VT probability estimates any time a WCT is recorded (i.e., ‘captured’) by a 12-lead ECG whether or not a baseline ECG has been recorded.

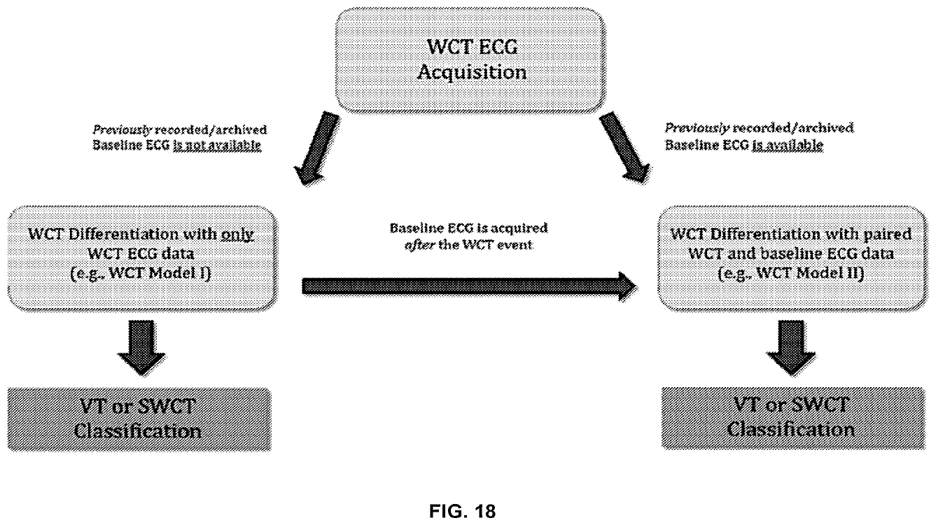

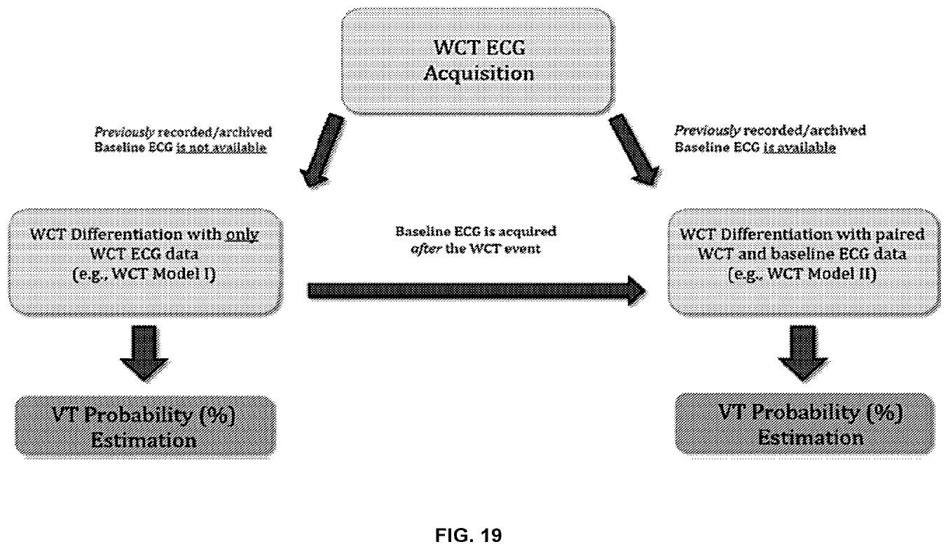

In addition, the use of WCT Model I to differentiate WCT ECG data can be coordinated with other high-performing models that require computerized data from paired WCT and baseline ECGs (FIGS. 18 and 19). For example, once a previously absent baseline ECG is recorded after the WCT event, the WCT Model I can cede its application to a more robust prediction model (e.g., WCT Model II) that makes use of ECG data provided by paired WCT and baseline ECGs. However, if a patient already possesses a digitally archived baseline ECG that is available for automated WCT differentiation algorithm application, VT or SWCT classification or VT probability estimation may be executed by more robust models that leverage paired WCT and baseline ECG comparisons, and thereby supersede the predictions attained by WCT Model I, which only analyzes computerized ECG data provided by the WCT itself.

The WCT differentiation methods disclosed herein transform computerized ECG data, routinely processed by ECG interpretation software programs, into novel engineered parameters that are integrated into binary classification models that use machine learning methods including, but not limited to logistic regression. An advantage of the disclosed WCT differentiation methods is that they provide clinicians with an impartial estimation of VT likelihood (i.e., 0.00% to 99.99% VT probability) using a procedure that functions independently of a clinician's ECG interpretation expertise or competency. Therefore, clinicians using the disclosed methods would be able to integrate automatically-provided VT probability estimates with the probabilistic influence of traditional WCT differentiation methods such as the Brugada algorithm, or other relevant clinical information such as a history of prior VT. In some aspects, the disclosed WCT differentiation methods are suitable for integration and/or implementation with commercially available ECG interpretation software platforms to more adeptly assist clinicians in distinguishing VT and SWCT accurately.

### Computing Systems and Devices

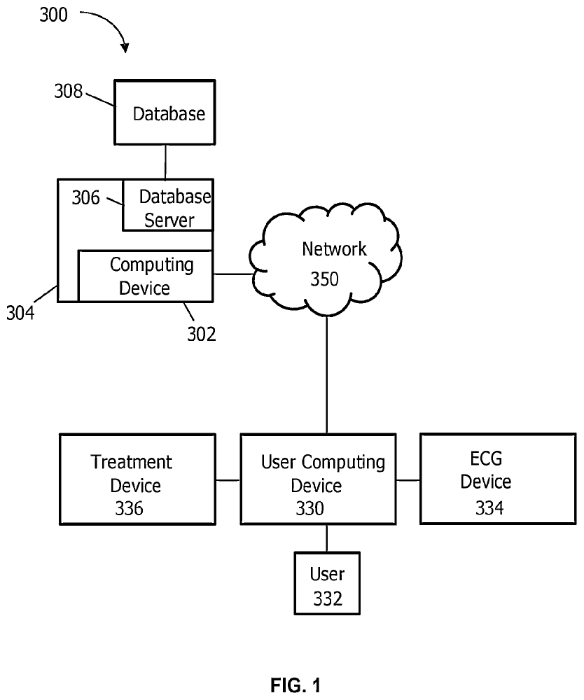

In various aspects, the disclosed method may be implemented using a computing system or computing device. FIG. 1 depicts a simplified block diagram of the system for implementing the computer-aided method of classifying a wide complex tachycardia (WCT) pattern in a subject as described herein. As illustrated in FIG. 1, the computing device 300 may be configured to implement at least a portion of the tasks associated with the disclosed methods of classifying the WCT patterns as described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

In some aspects, the system 300 may further include an ECG device 334 operatively coupled to the user computing device 330. As described herein, the ECG device 334 is configured to obtain and communicate ECG measurements from the heart of the subject to the user computing device 330. The ECG device 334 includes any of the suitable devices described herein including, but not limited to a 12-lead ECG device. In other aspects, the ECG device 334 may further provide for the transformation of the ECG measurements into at least one engineered parameter using automated data analysis software.

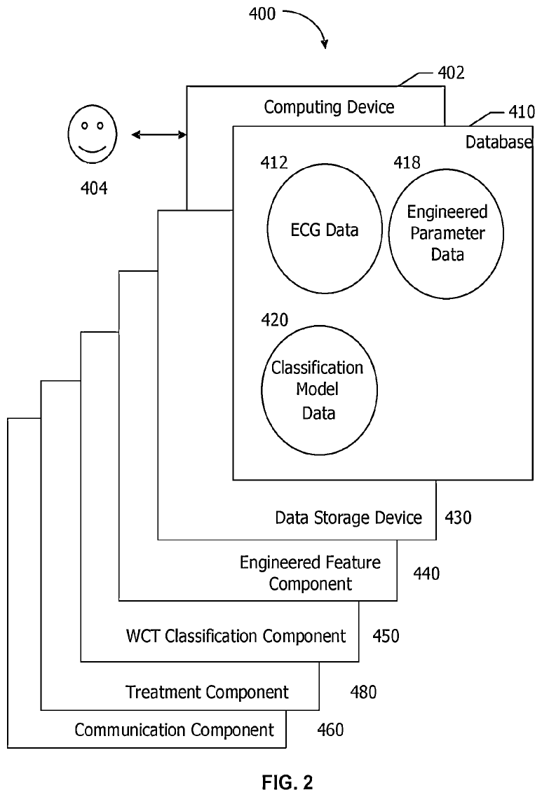

In other aspects, the computing device 302 is configured to perform a plurality of tasks associated with the disclosed computer-aided method of classifying a wide complex tachycardia (WCT) pattern in a subject. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes ECG data 412, engineered parameter data 418, and classification model data 420. Non-limiting examples of suitable ECG data 412 include any values of parameters defining the baseline ECG data and/or WCT ECG data measured by the ECG device. In some aspects, the ECG data 412 may include values of one or more engineered features computed by automated ECG data analysis software based on the ECG data as described herein.

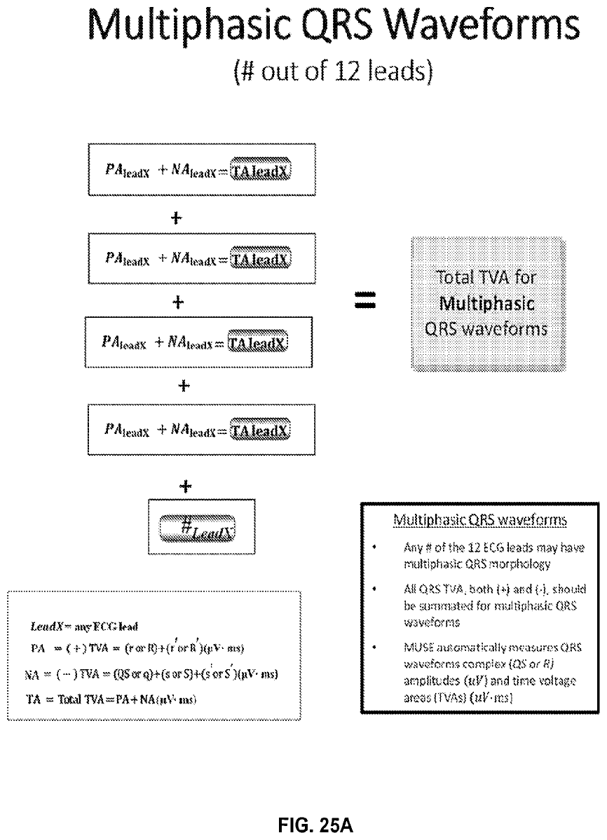

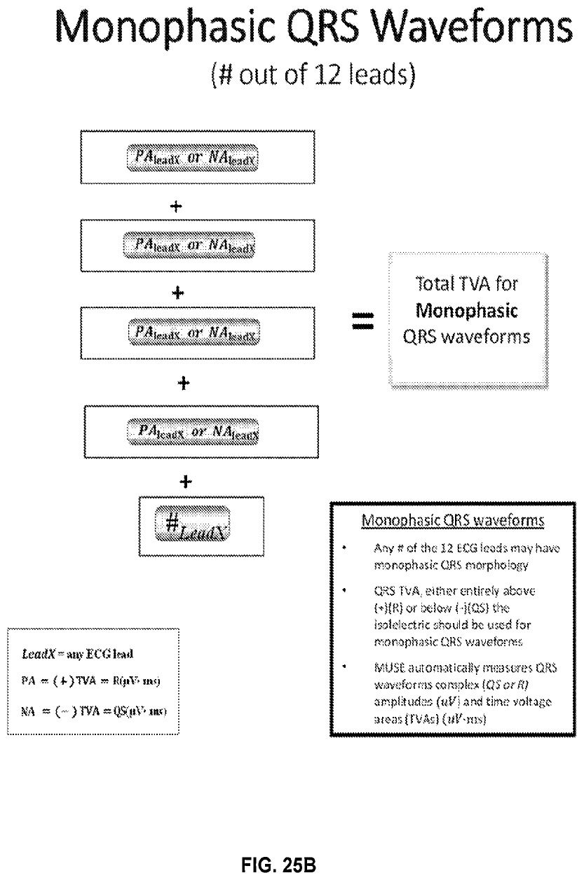

Non-limiting examples of engineered parameter data 418 include one or more parameters defining the transforms used to produce the various engineered features based on the ECG data as described herein. By way of non-limiting example, the engineered parameter data 418 may include various values used to define the calculation of the engineered parameters Monophasic TVA (FIG. 25B), Multiphasic TVA (FIG. 25A), and PMonoTVA (FIG. 7) as described herein. In other aspects, the engineered parameter data 418 may include values of the engineered parameters calculated as described herein, for use in classifying the WCT ECG pattern as described herein.

Non-limiting examples of the classification model data 420 include values defining the architecture and specific implementations of one or more of the machine learning models used to classify the WCT ECG based on the engineered parameter values as described herein. In some aspects, the classification model data 420 may include training data used to train the machine learning models as described herein. In additional aspects, the classification model data 420 may include the WCT classification produced by the machine learning model as described herein, treatment recommendations based on the WCT classifications as described herein, and the treatment rules used to produce the treatment recommendations as described herein.

Computing device 402 also includes a number of components that perform specific tasks. In the exemplary aspect, computing device 402 includes data storage device 430, engineered feature component 440, WCT classification component 450, treatment component 480, and communication component 460. Data storage device 430 is configured to store data received or generated by computing device 402, such as any of the data stored in database 410 or any outputs of processes implemented by any component of computing device 402.

In various aspects, the engineered feature component 440 implements the transformation of the WCT ECG data and/or baseline ECG data into the various engineered features as described herein. In some aspects, the engineered feature component 440 receives at least a portion of the engineered parameters from automated data analysis software implemented using the disclosed system 300 or by an additional device including, but not limited to the ECG device 334 (see FIG. 1). In various aspects, the WCT classification component 450 implements the training of the machine learning model and/or the classification of the WCT ECG pattern as a VT or an SWCT using the trained machine learning model as described herein. In various aspects, the treatment component 480 implements the transformation of the classification of the WCT ECG pattern into a treatment recommendation as described herein. In some aspects, the treatment component 480 may produce signals used to operate a treatment device including, but not limited to, the treatment device 336 which may be included as part of the system 300 (see FIG. 1).

Communication component 460 is configured to enable communications between computing device 402 and other devices (e.g. user computing device 330 shown in FIG. 1) over a network, such as network 350 (shown in FIG. 1), or a plurality of network connections using predefined network protocols such as TCP/IP (Transmission Control Protocol/Internet Protocol).

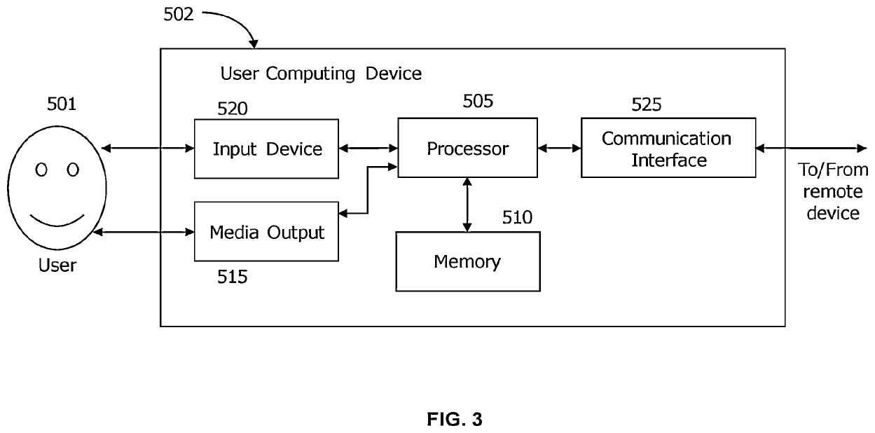

FIG. 3 depicts a configuration of a remote or user computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touch pad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

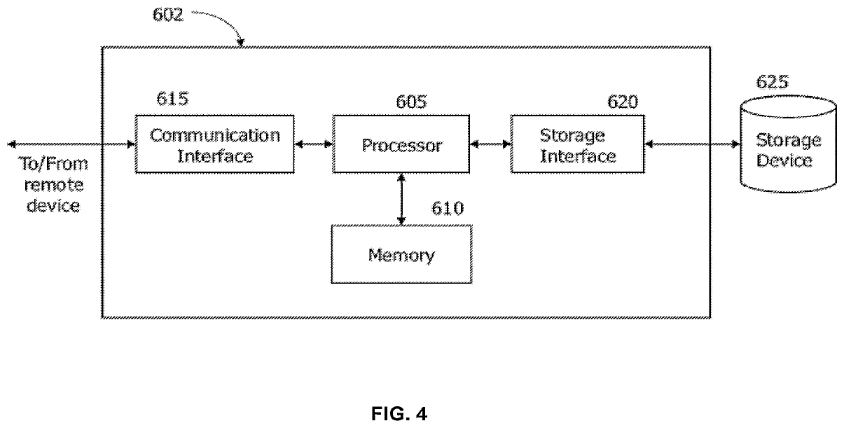

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Comparison of WCT Differentiation Models

To compare and evaluate several methods of WCT differentiation making use of a variety of differentiation models, the following experiments were conducted.

Parameters generated from WCT and baseline ECG data as described herein were used to derive, validate, and compare five different binary classification models: (i) logistic regression [LR], (ii) artificial neural network [ANN], (iii) Random Forests [RF], (iv) support vector machine [SVM]), and (v) ensemble learning (EL). The collective averages of VT probabilities achieved by each of the LR, ANN, RF, and SVM models based on the same WCT/ECG dataset were compared to evaluate the accuracy of each model.

### ECG Selection

Analyzed ECGs were acquired from genuine clinical settings at various hospital sites of the BJC HealthCare system. Each ECG was a standard 12-lead recording (paper speed: 25 mm/s and voltage calibration: 10 mm/mV) accessed from centralized data archives provided by a proprietary ECG interpretation software system (MUSE [GE Healthcare; Milwaukee, WI]).

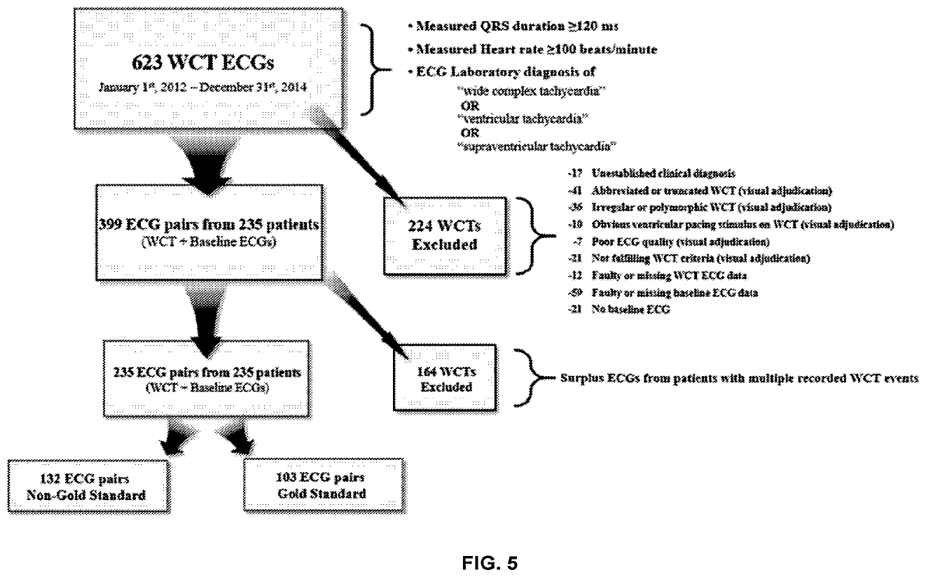

WCT ECGs were selected from consecutive patients who presented with WCT between Jan. 1, 2012 and Dec. 31, 2014 (FIG. 5). WCTs were required to satisfy standard WCT criteria (QRS duration ≥120 ms and ventricular rate ≥100 beats per minute) and possess an official ECG laboratory interpretation of (i) “ventricular tachycardia,” (ii) “supraventricular tachycardia,” or (iii) “wide complex tachycardia.” Baseline ECGs were defined as the first non-WCT rhythm recorded after the WCT event. Polymorphic WCTs and WCTs demonstrating grossly irregular atrioventricular conduction (e.g., atrial fibrillation or atrial flutter with variable atrioventricular block) were excluded. ECGs demonstrating truncated WCTs (e.g., a brief run of non-sustained VT) occurring within a dominant baseline heart rhythm (e.g., normal sinus rhythm) were not evaluated. If a WCT did not have a baseline ECG or definitive clinical diagnosis established by the patient's overseeing physician, it was excluded from further analysis. Among patients having multiple WCT events, the surplus of WCT ECGs occurring after the first WCT event was excluded (i.e., a single WCT and baseline ECG pair was evaluated per patient).

### Study Cohorts

A total of 235 patients (i.e., whole study cohort) presenting with paired WCT and baseline were analyzed. Of the 235 patients, 103 heart rhythm diagnoses (i.e., VT or SWCT) were established with a corroborating electrophysiology procedure (EP) or implantable intracardiac device recordings (i.e., gold standard cohort). Of the 235 patients, 132 heart rhythm diagnoses (i.e., VT or SWCT) were established without a corroborating EP or implantable intracardiac device recordings (i.e., non-gold standard cohort). Patient data acquisition and analysis were approved by the Human Research Protection Office of Washington University in St. Louis.

A total of 103 patients of the gold standard cohort were used to derive and validate five different binary classification models: (i) logistic regression [LR], (ii) artificial neural network [ANN], (iii) Random Forests [RF], (iv) support vector machine [SVM]), and (v) ensemble learning (EL)—the collective average of VT probabilities from the LR, ANN, RF, and SVM models. Each of the five models integrated nine engineered features derived from paired WCT and baseline ECGs and the WCT ECG alone: PMonoTVA (%), WCT QRS duration (ms), QRS duration change (ms), QRS axis change (°), T wave axis change (°), frontal PAC (%), horizontal PAC (%), frontal PTVAC (%), and horizontal PTVAC (%). Given its interrelatedness with PMonoTVA, PMonoAmp (%) was deliberately not integrated into the binary classification models.

### Heart Rhythm Diagnoses

Heart rhythm diagnoses (i.e., VT or SWCT) were established by the patient's supervising physician. Physicians responsible for clinical diagnoses were stratified according to a hierarchy of clinical expertise: (i) heart rhythm cardiologist, (ii) non-heart rhythm cardiologist, and (iii) non-cardiologist. Heart rhythm diagnoses were organized according to whether they were supported by a corroborating EP or implantable intracardiac device recordings (i.e., gold standard cohort vs. non-gold standard cohort).

### Computerized ECG Measurements

Standard computerized ECG measurements for WCT and baseline ECGs, including QRS duration (ms), QRS axis (°), and T wave axis (°), were automatically generated by GE Healthcare's MUSE ECG interpretation software. Computerized QRS amplitude (μV) and TVA (time-voltage area) (μV·ms) measurements of waveforms above (r/R and r′/R′) and below (q/QS, s/S, and s′/S′) the isoelectric baseline were automatically derived from the dominant QRS complex template within each lead of the 12-lead ECG (FIG. 6). Only amplitude and TVA measurements representative of QRS complex waveforms were analyzed. Measurements of the ventricular pacing stimuli or ECG artifact were excluded from our analysis.

### WCT Differentiation Parameters

Various engineered parameters disclosed herein were used to develop and assess the WCT differentiation models in these experiments. Engineering parameters included percent monophasic time-voltage area (PMonoTVA), percent monophasic amplitude (PMonoAmp), frontal percent amplitude change (FPAC), horizontal percent amplitude change (HPAC), frontal percent time-voltage area change (FPTVAC), horizontal percent time-voltage area change (HPTVAC), WCT QRS duration, baseline QRS duration, QRS duration change, QRS axis change, and T wave axis change.

### Statistical Analysis

All statistical analysis was performed using R Statistical Software (R Foundation for Statistical Computing, Vienna, Austria). Categorical variables were compared using Chi-square tests. Wilcoxon rank-sum tests were used to compare continuous variables. A two-tailed p-value of <0.05 was considered statistically significant.

Five modeling techniques (LR, ANN, RF, SVM, and EL) were used to derive and validate binary classification models for 103 patients of the gold standard cohort. To compare the models, the process was repeated 100 times, each time selecting a unique random sample of patients. Binary rhythm classification (i.e., VT or SWCT) was rendered according to a pre-specified VT probability partition of 50% (i.e., VT≥50% and SWCT<50%). The diagnostic performance (i.e., accuracy, sensitivity, specificity, and area under the curve [AUC]) were assessed according to their agreement with the heart rhythm diagnosis (i.e., VT or SWCT).

### Clinical Characteristics

Clinical characteristics of the derivation cohort are shown in Table 1. Among derivation cohort patients, the VT group included more patients with coronary artery disease, prior myocardial infarction, ongoing antiarrhythmic drug use, ischemic cardiomyopathy, and an implantable cardioverter-defibrillator (ICD). The SWCT group included more patients with an implanted pacemaker. The VT group comprised more patients with a severely depressed (≤30%) left ventricular ejection fraction (LVEF), whereas the SWCT group included more patients with a preserved (≥50%) LVEF. A majority of patients with VT diagnoses had a corroborating EP or intracardiac device recording. Conversely, a minority of SWCT patients had a corroborating EP or intracardiac device recording.

TABLE 1
Characteristics of the derivation cohort

| | | | |
|---|---|---|---|
| | SWCT | VT | |
| Derivation Cohort | (n = 242) | (n = 179) | p-value |

Diagnosing provider

| | | | | | |
|---|---|---|---|---|---|
| Cardiologist | 84 | (34.7) | 16 | (8.9) | <0.001 |
| Heart rhythm cardiologist | 92 | (38.0) | 155 | (86.6) | |
| Non-cardiologist | 66 | (27.3) | 8 | (4.5) | |

Patient Age

| | | | | |
|---|---|---|---|---|
| 70 | (15) | 66 | (14) | 0.006 |

Clinical Characteristics

| | | | | | |
|---|---|---|---|---|---|
| Coronary artery disease | 116 | (47.9) | 121 | (67.6) | <0.001 |
| Prior myocardial infarction | 63 | (26.0) | 102 | (57.0) | <0.001 |
| Prior cardiac surgery | 92 | (38.0) | 75 | (41.9) | 0.481 |
| Congenital heart disease | 16 | (6.6) | 12 | (6.7) | 1.0 |
| Anti-arrhythmic drug use | 36 | (14.9) | 97 | (54.2) | <0.001 |
| Ischemic cardiomyopathy | 38 | (15.7) | 89 | (49.7) | <0.001 |
| Non-ischemic cardiomyopathy | 53 | (21.9) | 54 | (30.2) | 0.07 |
| ICD | 17 | (7.0) | 112 | (62.6) | <0.001 |
| Pacemaker | 18 | (7.4) | 4 | (2.2) | 0.032 |

Left Ventricular Ejection Fraction (%)

| | | | | | |
|---|---|---|---|---|---|
| LVEF (>=50) | 140 | (57.9) | 48 | (26.8) | <0.001 |
| LVEF (49-31) | 48 | (19.8) | 60 | (33.5) | |
| LVEF (<=30) | 42 | (17.4) | 70 | (39.1) | |
| LVEF Unknown | 12 | (5.0) | 1 | (0.6) | |

Gold Standard Diagnosis

| | | | | | |
|---|---|---|---|---|---|
| Yes | 63 | (26.0) | 129 | (72.1) | <0.001 |

Numbers in parentheses are percent (%) of n. Patients having a gold standard diagnosis were those with a corroborating EP or implantable intracardiac device recordings. ECG, electrocardiogram; ICD, implantable cardioverter-defibrillator; SWCT, supraventricular tachycardia; VT, ventricular tachycardia.

### WCT Differentiation Parameters

Table 2 summarizes ECG engineered feature values across VT and SWCT groups. The VT group expressed greater PMonoTVA, WT QRS duration, QRS duration change, QRS axis change, T wave axis change, frontal PAC, horizontal PAC, frontal PTVAC, and horizontal PTVAC.

TABLE 2
WCT Differentiation Parameters

| | WCT | SWCT | VT | |
|---|---|---|---|---|
| | (n = 235) | (n = 158) | (n = 77) | p-value |
| PMonoA (%) | 21.4 | 10.8 | 49.7 | <0.001 |
| | [5.2, 50.8] | [0, 31.7] | [31.5, 72.0] | |
| PMonoTVA (%) | 23.55 | 11.65 | 55.63 | <0.001 |
| | [5.53, 58.36] | [0.00, 36.63] | [35.43, 74.24] | |
| Baseline QRS | 132.00 | 134.00 | 128.00 | 0.884 |
| duration (ms) | [112.00, 154.00] | [114.00, 152.00] | [110.00, 64.00] | |
| WCT QRS | 146.00 | 140.00 | 162.00 | <0.001 |
| Duration (ms) | [132.00, 162.00] | [128.00, 152.00] | [144.00, 186.00] | |
| QRS duration | 16.00 | 12.00 | 44.00 | <0.001 |
| change (ms) | [6.00, 44.00] | [4.50, 27.50] | [16.00, 62.00] | |
| QRS Axis | 23.00 | 12.50 | 89.00 | <0.001 |
| Change (°) | [8.00, 73.00] | [4.00, 34.50] | [45.00, 127.00] | |
| T Axis | 42.00 | 23.00 | 104.00 | <0.001 |
| Change (°) | [14.00, 103.00] | [9.00, 51.00] | [71.00, 142.00] | |
| Frontal | 52.35 | 29.72 | 110.25 | <0.001 |
| PAC (%) | [24.33, 96.93] | [18.22, 55.37] | [80.96, 149.12] | |
| Horizontal | 57.70 | 41.62 | 111.79 | <0.001 |
| PAC (%) | [36.80, 97.77] | [26.39, 67.39] | [74.31, 140.21] | |
| Frontal | 59.89 | 35.48 | 163.82 | <0.001 |
| PTVAC (%) | [29.03, 145.03] | [23.03, 66.63] | [127.80, 285.25] | |
| Horizontal | 72.45 | 54.29 | 168.47 | <0.001 |
| PTVAC (%) | [39.11, 153.79] | [30.92, 82.23] | [114.98, 238.57] | |

Displayed numbers represent mean values. Numbers in parentheses are interquartile ranges. Abbreviations: PMonoTVA; percent monophasic time voltage area; PAC, percent amplitude change, PTVAC, percent time-voltage area change; SWCT, supraventricular tachycardia; WCT, wide complex tachycardia; VT, ventricular tachycardia.

The median and proportional distributions of PMonoTVA among VT and SWCT groups are shown in FIG. 11. The median and proportional distributions of PMonoAmp among VT and SWCT groups are shown in FIG. 12.

The diagnostic performance metrics for each binary classification model subtype (i.e., LR, ANN, RF, SVM, and EL), when applied to the gold-standard cohort, are summarized in Table 3. Overall, each model subtype demonstrated similar diagnostic performance (i.e., accuracy, sensitivity, specificity, and AUC), except for the ANN which appeared to yield inferior performance across all indices of diagnostic performance.

TABLE 3
Diagnostic performance of WCT differentiation model subtypes

| | Accuracy | Sensitivity | Specificity | |
|---|---|---|---|---|
| | (%) | (%) | (%) | AUC |
| Logistic | 90.3 | 87.5 | 92.9 | 95.4 |
| Regression | (87.1-93.5) | (80.0-93.3) | (87.5-94.5) | (92.4-98.0) |
| Artificial | 83.9 | 84.6 | 84.4 | 91.4 |
| Neural | (80.6-87.1) | (77.6-89.5) | (76.1-91.1) | (87.5-94.5) |
| Network | | | | |
| Random | 88.7 | 92.0 | 88.9 | 97.0 |
| Forest | (87.1-93.5) | (85.7-93.8) | (83.3-93.4) | (95.3-98.0) |
| Support | 90.3 | 92.3 | 93.5 | 97.8 |
| Vector | (87.1-93.5) | (83.3-100.0) | (88.9-100.0) | (96.1-99.1) |
| Machine | | | | |
| Ensemble | 90.3 | 91.6 | 92.3 | 97.3 |
| Learner | (87.1-93.5) | (84.6-94.2) | (87.5-94.4) | (95.4-98.7) |

Displayed numbers represent mean values. Numbers in parentheses are interquartile ranges. AUC, area under the curve.

### Example 2—Comparison of Logistic Regression Models for WCT Differentiation

Patients of the whole study cohort (235 patients), gold-standard cohort (103 patients), and non-gold standard cohort (132 patients), as described herein in Example 1, were used to derive, validate, and compare two Logistic regression models: (i) a two engineered feature model (i.e., WCT Model I) composed of engineered features derived from computerized data of the WCT ECG alone (i.e., PMonoTVA [%] and WCT QRS duration [ms]) and (ii) a six engineered feature model (i.e., WCT Model II) comprised of independent engineered features derived from computerized data of the WCT and baseline ECGs (i.e., PMonoTVA [%], WCT QRS duration [ms], QRS axis change [°], T wave axis change [°], frontal PAC [%], and horizontal PAC [%]).

Two LR models (i.e., WCT Model I and WCT Model II) were fit using 5-fold cross-validation for the (i) whole study cohort, (ii) gold standard cohort, and (iii) non-gold standard cohort. WCT Model I was comprised of two engineered features (i.e., PMonoTVA [%] and WCT QRS duration [ms]) derived from WCT ECG data alone (FIG. 9). WCT Model II incorporated six engineered features (i.e., PMonoTVA [%], WCT QRS duration [ms], QRS axis change [° ], T wave axis change [°], frontal PAC [%], and horizontal PAC [%]) selected using backward elimination from all available engineered features able to be derived paired WCT and baseline ECGs and the WCT ECG alone (FIG. 10). Outlier values for each engineered feature were winsorized to diminish undue influence on model coefficients. Binary heart rhythm classification (i.e., VT or SWCT) was established using a pre-specified VT probability partition of 50% (i.e., VT≥50% and SWCT<50%). Performance metrics (i.e., accuracy, sensitivity, specificity, and AUC) for each model were assessed according to their agreement with the heart rhythm diagnosis (i.e., VT or SWCT). A comparison of the fit between the statistical models was completed using a Delong test. The Bonferroni method was used to adjust p-values for multiple comparisons.

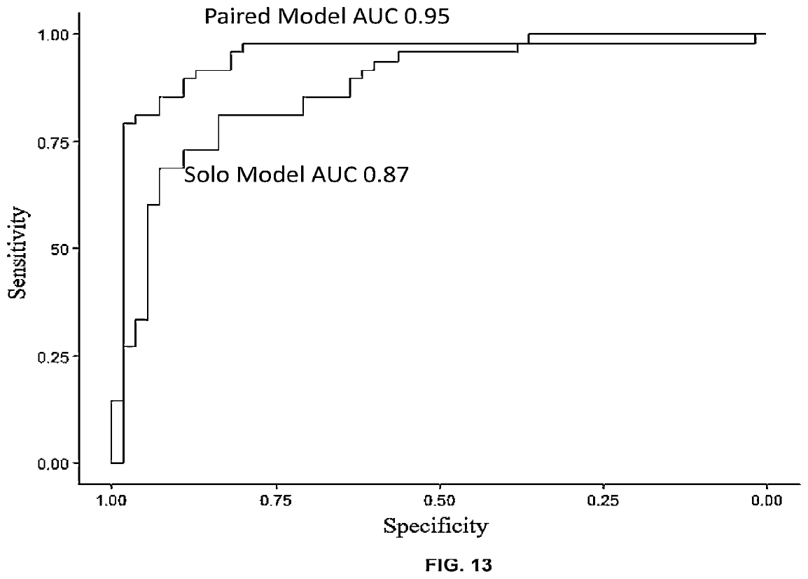

Diagnostic performance metrics (i.e., accuracy, sensitivity, specificity, and AUC) of the WCT Model I and WCT Model II across each patient cohort (i.e., whole study cohort, gold standard cohort, and non-gold standard cohort) are summarized in Table 4. Head-to-head comparisons of diagnostic performance between the WCT Model I and WCT Model II are presented in Table 5. The WCT Model II demonstrated superior overall accuracy, sensitivity, and AUC compared to WCT Model I across each study cohort. Conversely, the diagnostic specificity of the WCT Model I and WCT Model II was similar across each study cohort. A comparison of the receiver operating characteristic (ROC) curves of the WCT Model I (AUC 0.862) and WCT Model II (AUC 0.955) when applied to the whole study cohort is shown in FIG. 13.

TABLE 4
WCT Model I (solo) and WCT Model II
(paired) diagnostic performance

| | | Accuracy | Sensitivity | Specificity | |
|---|---|---|---|---|---|
| | Cohort | (%) | (%) | (%) | AUC |
| WCT | Whole Study | 79.9 | 59.4 | 89.9 | 0.862 |
| Model I | Gold Standard | 77.5 | 64.9 | 90.5 | 0.866 |
| | Non-Gold | 81.9 | 48.7 | 98.7 | 0.844 |
| | Standard | | | | |
| WCT | Whole Study | 88.4 | 83.3 | 91.0 | 0.955 |
| Model II | Gold Standard | 88.8 | 83.4 | 94.3 | 0.974 |
| | Non-Gold | 88.1 | 83.0 | 89.3 | 0.945 |
| | Standard | | | | |

Summary of Solo Model and Paired Model diagnostic performance across the gold standard and non-gold standard cohort. AUC, area under the receiver operator curve.

TABLE 5
Model comparison: WCT Model I vs. WCT Model II

| Model | | Accuracy | Sensitivity | Specificity | AUC |
|---|---|---|---|---|---|
| Comparison | Cohort | (p-value) | (p-value) | (p-value) | (p-value) |
| WCT | Whole | <0.001* | <0.001* | 1.000 | <0.001* |
| Model I | Study | | | | |
| vs | Gold | <0.001* | <0.001* | 0.921 | <0.001* |
| Model II | Standard | | | | |
| | Non-Gold | 0.001* | <0.001* | 1.000 | <0.001* |
| | Standard | | | | |

The overall accuracy of the WCT Model I did not differ across gold standard and non-gold standard study cohorts (p=0.132). Similarly, the overall accuracy of the WCT Model II did not differ across gold standard and non-gold standard study cohorts (p=1.000).

What is claimed is:

1. A computer-aided method of classifying a wide complex tachycardia (WCT) pattern of a subject, the method comprising:
a. receiving, using a computing device, WCT ECG data indicative of the WCT pattern;
b. transforming, using the computing device, the WCT ECG data into at least one engineered feature; wherein the at least one engineered feature is selected from a percent monophasic time-voltage area (PMonoTVA), a percent monophasic amplitude (PMonoAmp), a wide complex tachycardia (WCT) QRS duration, and any combination thereof;
c. transforming, using the computing device, the at least one engineered feature into an assigned classification of the WCT pattern using a machine learning model, wherein the classification of the WCT pattern is selected from a ventricular tachycardia (VT), a supraventricular wide complex tachycardia (SWCT), a probability of a VT, a probability of an SWCT, and any combination thereof; and
d. transforming the assigned classification of the WCT pattern into a treatment recommendation using at least one treatment rule, wherein the treatment rule is selected from:
i. recommending a shock delivery to the heart of the subject if the assigned classification is VT; or
ii. recommending no shock delivery if the assigned classification is SWCT.

2. (canceled)

3. The method of claim 1, wherein receiving the WCT ECG data indicative of the WCT pattern comprises receiving WCT ECG data from an ECG device comprising a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automatic implantable cardioverter defibrillator (AICD), an automated external defibrillator (AED), and any combination thereof.

4. (canceled)

5. (canceled)

6. The method of claim 1, wherein transforming the WCT ECG data into the at least one engineered feature further comprises:
a. transforming the WCT ECG data into the WCT QRS duration using automated data analysis software and receiving, using the computing device, the wide complex tachycardia (WCT) QRS duration from the automated data analysis software;
b. transforming the WCT ECG data into the PMonoTVA using a first transform comprising:
$$PMonoTVA = \frac{( Monophasic TVA )}{( Monophasic TVA ) + ( Multiphasic TVA )} \times 100$$
wherein Monophasic TVA comprises a summation of all QRS time-voltage areas from all monophasic QRS complexes from the ECG data and Multiphasic TVA comprises a summation of all QRS time-voltage areas from all multiphasic QRS complexes from the ECG data;
c. transforming the WCT ECG data into the PMonoAmp using a second transform comprising:
$$PMonoAmp = \frac{( Monophasic amplitude )}{( Monophasic amplitude ) + ( Multiphasic amplitude )} \times 100$$
wherein Monophasic amplitude comprises a summation of all QRS amplitudes from all monophasic QRS complexes from the ECG data and Multiphasic amplitude comprises a summation of all QRS amplitudes from all multiphasic QRS complexes from the ECG data; and
d. any combination thereof.

7. (canceled)

8. (canceled)

9. The method of claim 6, wherein
transforming, using the computing device, the at least one engineered feature into a classification of the WCT pattern using a machine learning model further comprises using a machine learning model comprising one of a logistic regression model, an artificial neural network, a random forest model, and a support vector machine.

10. (canceled)

11. The method of claim 10, wherein using the machine learning model comprising the logistic regression model further comprises:
a. transforming the at least one engineered feature into a weighted sum of predictors X_{β} using the equation:
$${X}_{β} = {β}_{0} + {β}_{1} {X}_{1} + {β}_{2} {X}_{2}$$
wherein β₀, β₁, and β₂ are constant weighting factors, X₁ is PMonoTVA, and X₂ is WCT QRS Duration; and
b. calculating the probability of the VT (P_{VT}) using the equation:
$${P}_{VT} = \frac{{e}^{{X}_{β}}}{1 + {e}^{{X}_{β}}} .$$

12. The method of claim 11, further comprising:
a. receiving, using the computing device, baseline ECG data indicative of a baseline cardiac pattern; and
b. transforming, using the computing device, the baseline ECG data and the WCT ECG data into at least one additional engineered feature selected from a QRS Axis change, a T Axis change, a frontal percent time-voltage area change (PTVAC), a Horizontal PTVAC, a frontal percent amplitude change (PAC), a horizontal PAC, and any combination thereof.

13. The method of claim 12, wherein transforming the baseline ECG data and the WCT ECG data into the at least one additional engineered feature further comprises:
a. transforming the WCT ECG data into a WCT QRS axis angle and the baseline ECG data into a baseline QRS axis angle using automated data analysis software, and subtracting, using the computing device, the baseline QRS axis angle from the WCT QRS axis angle to obtain the QRS Axis change;
b. transforming the WCT ECG data into a WCT T axis angle and the baseline ECG data into a baseline T axis angle using automated data analysis software, and subtracting, using the computing device, the baseline T axis angle from the WCT T axis angle to obtain the T Axis change;
c. transforming the WCT ECG data into a WCT Frontal percent amplitude (PA) and the baseline ECG data into a baseline Frontal PA using automated data analysis software, and subtracting, using the computing device, the baseline Frontal PA from the WCT Frontal PA to obtain the Frontal PAC; and
d. transforming the WCT ECG data into a WCT Horizontal percent amplitude (PA) and the baseline ECG data into a baseline Horizontal PA using automated data analysis software, and subtracting, using the computing device, the baseline Horizontal PA from the WCT Horizontal PA to obtain the Horizontal PAC.

14. The method of claim 13, wherein using the logistic regression model further comprises:
a. transforming the at least one engineered feature into a weighted sum of predictors X_{β} using the equation:
$${X}_{β} = {β}_{0} + {β}_{1} {X}_{1} + {β}_{2} {X}_{2} + {β}_{3} {X}_{3} + {β}_{4} {X}_{4} + {β}_{5} {X}_{5} + {β}_{6} {X}_{6}$$
wherein β₀, β₁, β₂, β₃, β₄, β₅, and β₆ are constant weighting factors, X₁ is PMonoTVA, X₂ is WCT QRS Duration, X₃ is QRS Axis change, X₄ is T Axis change, X₅ is Frontal PAC, and X₆ is Horizontal PAC; and
b. calculating the probability of the VT (P_{VT}) using the equation:
$${P}_{VT} = \frac{{e}^{{X}_{β}}}{1 + {e}^{{X}_{β}}} .$$

15. The method of claim 14, wherein transforming the at least one engineered feature into the classification of the WCT pattern further comprises:
a. assigning the classification of VT if P_{VT} is at least equal to a predetermined threshold value; and
b. assigning the classification of SWCT if P_{VT} is less than the predetermined threshold value.

16. (canceled)

17. The method of claim 15, wherein the predetermined threshold value comprises one of 1%, 10%, 25%, 50%, 75%, 90%, 95%, and 99%.

18. (canceled)

19. (canceled)

20. A system for classifying a wide complex tachycardia (WCT) pattern of a subject, the system comprising a computing device comprising at least one processor, the at least one processor configured to:
a. receive WCT ECG data indicative of the WCT pattern;
b. transform the WCT ECG data into at least one engineered feature; wherein the at least one engineered feature is selected from a percent monophasic time-voltage area (PMonoTVA), a percent monophasic amplitude (PMonoAmp), a wide complex tachycardia (WCT) QRS duration, and any combination thereof; and
c. transform the at least one engineered feature into an assigned classification of the WCT pattern using a machine learning model, wherein the classification of the WCT pattern is selected from a ventricular tachycardia (VT), a supraventricular wide complex tachycardia (SWCT), a probability of a VT, a probability of an SWCT, and any combination thereof; and
d. transform the assigned classification of the WCT pattern into a treatment recommendation using at least one treatment rule, wherein the treatment rule is selected from:
i. recommending a shock delivery to the heart of the subject if the assigned classification is VT; or
ii. recommending no shock delivery if the assigned classification is SWCT.

21. (canceled)

22. The system of any one of claim 20, wherein
the ECG device comprises one of a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automated external defibrillator (AED), an automatic implantable cardioverter defibrillator (AICD), and any combination thereof.

23. (canceled)

24. (canceled)

25. The system of claim 20, wherein the at least one processor is further configured to:
a. receive at least a portion of the engineered features from automated data analysis software configured to transform the WCT ECG data into the portion of the engineered features;
b. transform the ECG data into PMonoTVA using a first transform comprising:
PMonoTVA=(Monophasic TVA)/(Monophasic TVA)+(Multiphasic TVA)×100
wherein Monophasic TVA comprises a summation of all QRS time-voltage areas from all monophasic QRS complexes from the ECG data and Multiphasic TVA comprises a summation of all QRS time-voltage areas from all multiphasic QRS complexes from the ECG data;
c. transform the ECG data into PMonoAmp using a second transform comprising:
$$PMonoAmp = \frac{( Monophasic amplitude )}{( Monophasic amplitude ) + ( Multiphasic amplitude )} \times 100$$
wherein Monophasic amplitude comprises a summation of all QRS amplitudes from all monophasic QRS complexes from the ECG data and Multiphasic amplitude comprises a summation of all QRS amplitudes from all multiphasic QRS complexes from the ECG data; and
d. any combination thereof.

26. (canceled)

27. (canceled)

28. The system of claim 26, wherein the at least one processor is further configured to transform the least one engineered feature into a classification of the WCT pattern using a machine learning model comprising one of a logistic regression model, an artificial neural network, a random forest model, and a support vector machine.

29. (canceled)

30. The system of claim 28, wherein the at least one processor is further configured to transform the least one engineered feature into a classification of the WCT pattern using the logistic regression model by:
a. transforming the at least one engineered feature into a weighted sum of predictors X_{β} using the equation:
$${X}_{β} = {β}_{0} + {β}_{1} {X}_{1} + {β}_{2} {X}_{2}$$
wherein β₀, β₁, and β₂ are constant weighting factors, X₁ is PMonoTVA, and X₂ is WCT QRS Duration; and
b. calculating the probability of the VT (P_{VT}) using the equation:
$${P}_{VT} = \frac{{e}^{{X}_{β}}}{1 + {e}^{{X}_{β}}} .$$

31. The system of claim 30, wherein the at least one processor is further configured to:
a. receive baseline ECG data indicative of a baseline cardiac pattern; and
b. transform the baseline ECG data and the WCT ECG data into at least one additional engineered feature selected from a QRS Axis change, a T Axis change, a frontal percent time-voltage area change (PTVAC), a Horizontal PTVAC, a frontal percent amplitude change (PAC), a horizontal PAC, and any combination thereof.

32. The system of claim 31, wherein the at least one processor is further configured to:
a. transform the WCT ECG data into a WCT QRS axis angle and the baseline ECG data into a baseline QRS axis angle using automated data analysis software, and subtracting, using the computing device, the baseline QRS axis angle from the WCT QRS axis angle to obtain the QRS Axis change;
b. transform the WCT ECG data into a WCT T axis angle and the baseline ECG data into a baseline T axis angle using automated data analysis software, and subtracting, using the computing device, the baseline T axis angle from the WCT T axis angle to obtain the T Axis change;
c. transform the WCT ECG data into a WCT Frontal percent amplitude (PA) and the baseline ECG data into a baseline Frontal PA using automated data analysis software, and subtracting, using the computing device, the baseline Frontal PA from the WCT Frontal PA to obtain the Frontal PAC; and
d. transform the WCT ECG data into a WCT Horizontal percent amplitude (PA) and the baseline ECG data into a baseline Horizontal PA using automated data analysis software, and subtracting, using the computing device, the baseline Horizontal PA from the WCT Horizontal PA to obtain the Horizontal PAC.

33. The system of claim 32, wherein the at least one processor is further configured to transform the least one engineered feature into a classification of the WCT pattern using the logistic regression model by:
a. transforming the at least one engineered feature into a weighted sum of predictors X_{β} using the equation:
$${X}_{β} = {β}_{0} + {β}_{1} {X}_{1} + {β}_{2} {X}_{2} + {β}_{3} {X}_{3} + {β}_{4} {X}_{4} + {β}_{5} {X}_{5} + {β}_{6} {X}_{6}$$
wherein β₀, β₁, β₂, β₃, β₄, β₅, and β₆ are constant weighting tactors, X₁ is PMonoTVA, X₂ is WCT QRS Duration, X₃ is QRS Axis change, X₄ is T Axis change, X₅ is Frontal PAC, and X₆ is Horizontal PAC; and
b. calculating the probability of the VT (P_{VT}) using the equation:
$${P}_{VT} = \frac{{e}^{{X}_{β}}}{1 + {e}^{{X}_{β}}} .$$

34. The system of claim 33, wherein the at least one processor is further configured to transform the least one engineered feature into a classification of the WCT pattern using the logistic regression model by:
a. assigning the classification of VT if P_{VT} is at least equal to a predetermined threshold value; and
b. assigning the classification of SWCT if P_{VT} is less than the predetermined threshold value.

35. (canceled)

36. (canceled)

37. (canceled)

38. (canceled)

39. The system of claim 35, wherein the system further comprises the ECG device, a treatment device, and any combination thereof operatively coupled to the computing device.

40. (canceled)
